(19) United States
(12) Patent Application Publication
(10) Pub. No.: US 2024/0318143 A1
(43) Pub. Date: Sep. 26, 2024

(54) POST-PROCESSING OF BIOLOGICAL SCAFFOLDS

(71) Applicant: Lung Biotechnology PBC, Silver Spring, MD (US)

(72) Inventors: Victor Hernandez Gordillo, Cambridge, MA (US); Aman Kaur, Manchester, NH (US); Barbara Nsiah, Manchester, NH (US); Derek Morris, Bedford, NH (US); Luis Alvarez, Lexington, MA (US)

(73) Assignee: Lung Biotechnology PBC, Silver Spring, MD (US)

(21) Appl. No.: 18/611,429

(22) Filed: Mar. 20, 2024

Related U.S. Application Data
(60) Provisional application No. 63/453,567, filed on Mar. 21, 2023.

(51) Int. Cl. C12N 5/071; C07K 1/04; C07K 7/06; C07K 7/08; C07K 14/00

(52) U.S. Cl. CPC C12N 5/0688; C07K 1/042; C07K 7/06; C07K 7/08; C07K 14/00; C12N 2513/00; C12N 2533/50; C12N 2537/10

(57) ABSTRACT

A method of modifying a surface of a three-dimensional (3D) article includes immersing at least one part of the 3D article in a buffered solution of functionalized peptides, allowing reaction between the functionalized peptides and reactive groups on the surface of the 3D article; and washing the at least one part of the 3D article to remove unreacted functionalized peptides. The surface-modified 3D article includes a plurality of peptides covalently bonded to the surface of the 3D article via a cysteine bridge. The surface-modified 3D article can be used as a scaffold for the formation of biological tissue or bodily implants.

### CROSS-REFERENCE TO RELATED APPLICATIONS

The application claims the benefit of and priority to U.S. Provisional Patent Application No. 63/453,567, filed Mar. 21, 2023, which is incorporated by reference herein in its entirety for any and all purposes.

### SEQUENCE LISTING

The instant application contains a Sequence Listing which has been submitted electronically in XML format and is hereby incorporated by reference in its entirety. Said XML copy, created on Apr. 17, 2024, is named 080618-2395_SL.xml and is 14,703 bytes in size.

### FIELD OF THE INVENTION

The present disclosure concerns an apparatus and method for modifying surfaces of solid three-dimensional (3D) articles of manufacture. More particularly, the present disclosure concerns a method of modifying surfaces of 3D articles with peptides that promote biological cell interaction with the 3D articles.

### BACKGROUND

Three dimensional (3D) printers used to form 3D articles are in rapidly increasing use. One class of 3D printers includes stereolithography printers having a general principle of operation including the selective curing and hardening of radiation curable (photocurable) liquid inks. A typical stereolithography system includes a liquid tank holding the photocurable ink, a movement mechanism coupled to a support surface, and a controllable light engine. The stereolithography system forms a three-dimensional (3D) article of manufacture by selectively curing layers of the photocurable ink over the support surface.

The 3D articles formed using 3D printers can be used as tissue scaffolds for the formation of artificial tissue or bodily implants. Tissue scaffolds are useful for tissue engineering because they can mimic the extracellular matrix of the native tissue. These scaffolds can allow cell attachment and migration, deliver, and retain cells and biochemical factors, enable diffusion of vital cell nutrients and expressed products, and exert certain mechanical and biological influences to modify the behavior of the cell phase.

One particular challenge is initiating cell attachment onto the scaffolds. Some scaffolds, including those formed by selectively curing layers of the photocurable ink, can have synthetic surfaces that are not compatible with cells, resulting in poor cell attachment. A need exists for facilitating cell interaction with 3D articles, including printed 3D articles.

### SUMMARY

An aspect of the disclosure is a method of modifying a surface of a three-dimensional (3D) article. The surface of the 3D article can have a plurality of reactive groups. The method can include immersing at least one part of the 3D article in a buffered solution of functionalized peptides and allowing reaction between the functionalized peptides and the reactive groups on the surface of the 3D article.

In some embodiments, the method can also include washing at least one part of the 3D article to remove unreacted functionalized peptides. The reactive groups on the surface of the 3D article may be thiol-reactive groups. The functionalized peptides may include thiol-functionalized peptides.

In some embodiments, the buffered solution may have a pH of about 7.5 to about 10. The concentration of functionalized peptides may be from about 0.02 μmol per cm² on the surface of the 3D article to about 8.00 μmol per cm² on the surface of the 3D article. At least one of the functionalized peptides may bind to or have an affinity for at least one of syndecan, integrin, fibronectin, collagen IV, and laminin. At least one of the functionalized peptides may include binding domains for integrin and syndecan. Preferred functionalized peptides may include at least one amino acid sequence selected from the group consisting of CGRDRGDSPY (SEQ ID NO: 1), PHSRNGGGK(GGGERCG)GGRGDSPY (SEQ ID NO: 2) (GGGERCG disclosed as SEQ ID NO: 3), GCREKKRKRLQVQLSIRT (SEQ ID NO: 4), GCREIKVAV (SEQ ID NO: 5), GCREKKTLQPVYEYMVGV (SEQ ID NO: 6), GCREISAFLGIPFAEPPMGPRRFLPPEPKKP (SEQ ID NO: 7), and GGYGGGPG(GPP)5GFOGER(GPP)5GPC (SEQ ID NO: 8). The reactive groups comprise at least one of acrylate, thiol, maleimide, vinyl sulfone, norbornene, acrylamide, acrylonitrile, or methacrylate. The 3D article can be a lung scaffold or a portion thereof.

In some embodiments, the method may include forming the 3D article using 3D printing before immersing. The method may include contacting the 3D article with at least one type of cell that has affinity for the functionalized peptides. The method may include immersing the 3D article in cell media prior to contacting with the at least one cell type. Contacting the 3D article with the at least one cell type may include immersing the 3D article in a suspension of the cells for at least 1.5 hours. Contacting the 3D article with the at least one cell type may include intermittently perfusing the 3D article with a flowing suspension of cells at a flow rate of about 2 mL per minute to about 8 mL per minute. Contacting the 3D article with the at least one cell type may include constantly or intermittently perfusing the 3D article with a flowing suspension of cells.

Another aspect of the disclosure is a three-dimensional (3D) article including a polymer scaffold having a surface, and a plurality of peptides covalently bonded to the surface of the polymer scaffold via a cysteine bridge. At least some of the plurality of peptides bind to or have an affinity for at least two of syndecan, integrin, fibronectin, collagen IV, or laminin.

In some embodiments, the 3D article includes a plurality of biological cells disposed on the surface of the polymer scaffold. The biological cells may include primary adult lung cells. The biological cells may include induced pluripotent stem cells. The plurality of peptides may be bonded to the surface of the polymer scaffold via a thiol-acrylate Michael addition reaction. The polymer scaffold may be formed with 3D printing and acrylate groups that participate in the Michael addition reaction may have been formed at the surface of the polymer scaffold as part of the 3D printing. The polymer scaffold may include a lung scaffold.

Another aspect of the disclosure is a method of forming a three-dimensional (3D) article. The method includes forming a lung scaffold using 3D printing, the 3D printing forming a plurality of acrylate groups on an outer surface of the lung scaffold; immersing at least part of the lung scaffold in a buffered solution suspension of thiol-functionalized peptides that bind to or have an affinity for at least two of syndecan, integrin, fibronectin, collagen IV, or laminin, the buffered solution suspension having a pH of about 7.5 to about 10; washing the lung scaffold to remove unreacted thiol-functionalized peptides; after washing, conditioning the lung scaffold with cell media; and after conditioning, seeding biological cells on the outer surface of the lung scaffold using at least one of a static suspension of the biological cells or a flowing suspension of the biological cells.

### BRIEF DESCRIPTION OF THE FIGURES

FIG. 1 is a schematic diagram of an embodiment of a three-dimensional (3D) printed tissue scaffold.

FIG. 2 is a schematic diagram of a reaction scheme used to functionalize the surface of a tissue scaffold.

FIG. 3 is a flowchart depicting an embodiment of a method of functionalizing the surface of a tissue scaffold.

FIG. 4 is a flowchart depicting an embodiment of a method of culturing biological cells on a surface-functionalized tissue scaffold.

FIG. 5 shows graphs comparing cell coverage over time of surface-functionalized tissue scaffold.

FIG. 6 are stained disk scans of SAEC attachment and proliferation at day 11 on bioink modified with different peptide combinations prior to cellulation. The scan represents about 10 mm diameter of culture surface.

FIGS. 7A-7S illustrate PAEC morphology after 24 hours on discs surface modified with different peptides at a concentration of 2 μmol/cm². P represents PHSRNKRGD (SEQ ID NO: 9) peptide, G represents GFOGER (SEQ ID NO: 10) peptide, A represents AG73 peptide, B represents BM-Binder peptide, and F represents FN-Binder peptide. FIG. 7A are stained disk scans of PAECs cultured on 6 different surface modification peptides after 24 hours. FIGS. 7B-7S are graphs of FIJI analysis of circularity and roundness frequency distribution of the PAECs cultured on 6 different surface modification peptides after 24 hours.

FIGS. 8A-8G illustrate PAEC morphology after 24 hours on discs surface modified with various peptides at 2 μmol/cm² concentration. FIG. 8A are stained disk scans of PAECs cultured on 6 different surface modification peptides. Figure discloses “GFOGER” as SEQ ID NO: 10. FIGS. 8B-8G are graphs of FIJI analysis of circularity frequency distribution.

FIGS. 9A-9G illustrate PAEC morphology after 24 hours on discs surface modified with various peptides at 2 μmol/cm² concentration adjusted to the volume used in the PGABF′ condition. FIG. 9A are stained disk scans of PAECs cultured on 6 different surface modification peptides. Figure discloses “GFOGER” as SEQ ID NO: 10. FIGS. 9B-9G are graphs of FIJI analysis of circularity frequency distribution.

FIGS. 10A-10G illustrate PAEC morphology after 24 hours on discs surface modified with various peptides at 0.2 μmol/cm² concentration. FIG. 10A are stained disk scans of PAECs cultured on 6 different surface modification peptides. Figure discloses “GFOGER” as SEQ ID NO: 10. FIGS. 10B-10G are graphs of FIJI analysis of circularity frequency distribution.

FIG. 11 are images of induced pluripotent stem cell-derived endothelial cells (IPS-ECs) seeded at 50,000/cm² with 4 μMol, 0.2 μMol, and 0.02 μMol concentrations of PABF′ surface modification at day 1 and day 4 after cell seeding.

### DETAILED DESCRIPTION OF THE PREFERRED EMBODIMENTS

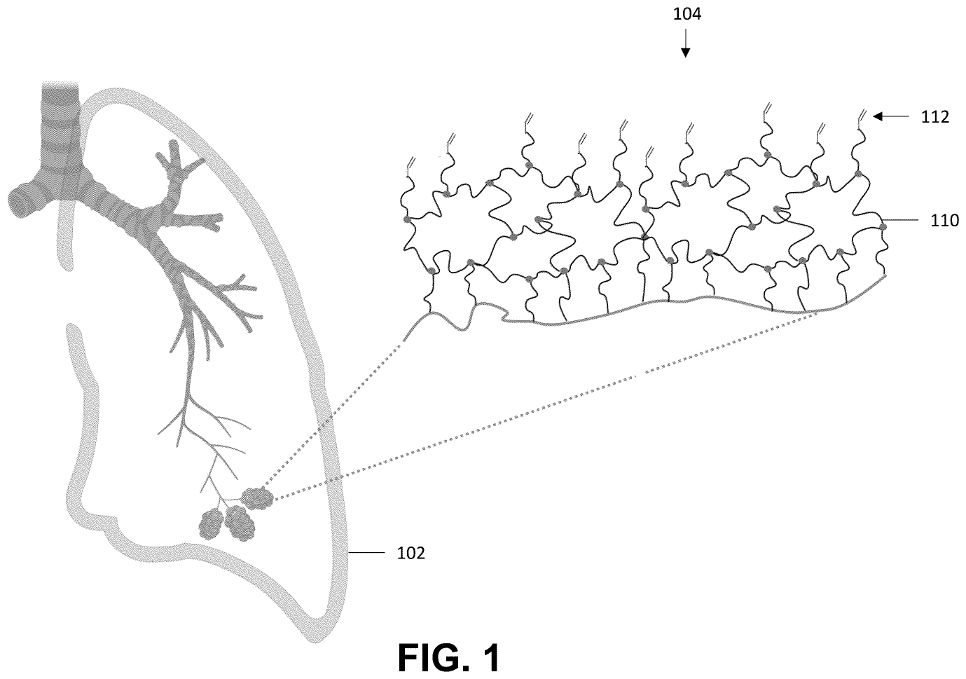

FIG. 1 is a schematic diagram of an embodiment of a three-dimensional (3D) article of manufacture. The 3D article is intended to act as a biological tissue scaffold 102 for the formation of artificial tissue or bodily implants. However, the 3D article as formed has a surface that is not ideal for forming adequate interactions with biological cells. The systems and methods of the present disclosure can be used to modify the surface of the 3D article so that biological cells can be seeded and cultured on the 3D article.

The tissue scaffolds serve as cell bioreactors that may be permeable to gas and/or fluid. The tissue scaffolds may mimic the extracellular matrix of the native tissue. The tissue scaffolds can include channels that model airways and/or vasculature. As a note, descriptions of the surfaces of tissue scaffolds may include the surfaces of any internal channels within the tissue scaffold.

The tissue scaffolds disclosed herein correspond to biological structures and/or functions. For example, the tissue scaffold can correspond to the structure or function of the pancreas, kidney, lung, liver, bone, or any organ which is made up of similar repeating structural units. The tissue scaffold does not necessarily need to be identical in structure or function to the corresponding naturally occurring biological structure or function. In some embodiments, the tissue scaffold 102 can be based on a model of the corresponding biological structure or function, and the tissue scaffold can comprise repeating structural or functional units of the corresponding organ. Some embodiments include tissue scaffolds corresponding to human lungs (as shown in FIG. 1) and kidneys. Functions of the tissue scaffold may include allowing cell attachment and migration, delivering, and retaining cells and biochemical factors, enabling diffusion of vital cell nutrients and expressing products, and/or exerting certain mechanical and biological influences to modify the behavior of the cell phase.

The tissue scaffold 102 may be formed using 3D printing. One class of 3D printers that may be used to form the tissue scaffold 102 include stereolithography printers. A stereolithography system forms the 3D article by selectively curing layers of a photocurable ink using selective exposure to certain wavelengths of light. The photocurable ink includes molecular chains terminated with chemical groups that are activated upon exposure to certain wavelengths of light. As an example, the photocurable ink may be selectively exposed to ultraviolet (UV) light to activate the terminal chemical groups and initiate radical polymerization of the photocurable ink, where the molecular chains join together to form the solidified 3D article. Other classes of 3D printers may be used to form the 3D article 102, including fused deposition modeling and selective laser sintering.

In addition to depicting the tissue scaffold 102 as a whole, FIG. 1 includes an inset 104 depicting a simplified schematic of the molecular structure at the surface of the tissue scaffold 102. The subsurface 110 of the tissue scaffold 102 is made of cross-linked polymers (e.g., formed from polymerization of a photocurable ink). The surface 112 of the tissue scaffold is populated with cross-linked polymer and a plurality of pendant chemical groups. The pendant chemical groups may result from the 3D printing process used to form the tissue scaffold 102.

In embodiments where the tissue scaffold 102 is formed using stereolithography 3D printing, the pendant chemical groups at the surface 112 include chemical groups originating from the photocurable ink (e.g., due to incomplete polymerization). As an example, the photocurable ink may include short carbon chains terminated with acrylate groups, and the resulting tissue scaffold made using this photocurable ink predominantly includes acrylate groups at the surface of the resulting tissue scaffold.

The pendant chemical groups at the surface 112 of the tissue scaffold may include acrylate groups, thiol groups, maleimide groups, vinyl sulfone groups, norbornene groups, acrylamide groups, acrylonitrile groups, and/or methacrylate groups. In some embodiments, a single type of pendant chemical group is predominant on the surface 112. In other embodiments, multiple types of chemical groups are substantially present on the surface 112. As an example, the dominant type of pendant chemical group at the surface is an acrylate.

The surface 112 of the tissue scaffold 102, with its cross-linked polymer and pendant chemical groups, often interacts poorly with biological cells. This poor interaction can make it difficult to seed and culture cells on the surface of the tissue scaffold 102. To promote cell attachment, the scaffolds can be post processed with a surface modification to allow cells to interact with the scaffold surface in the desired way.

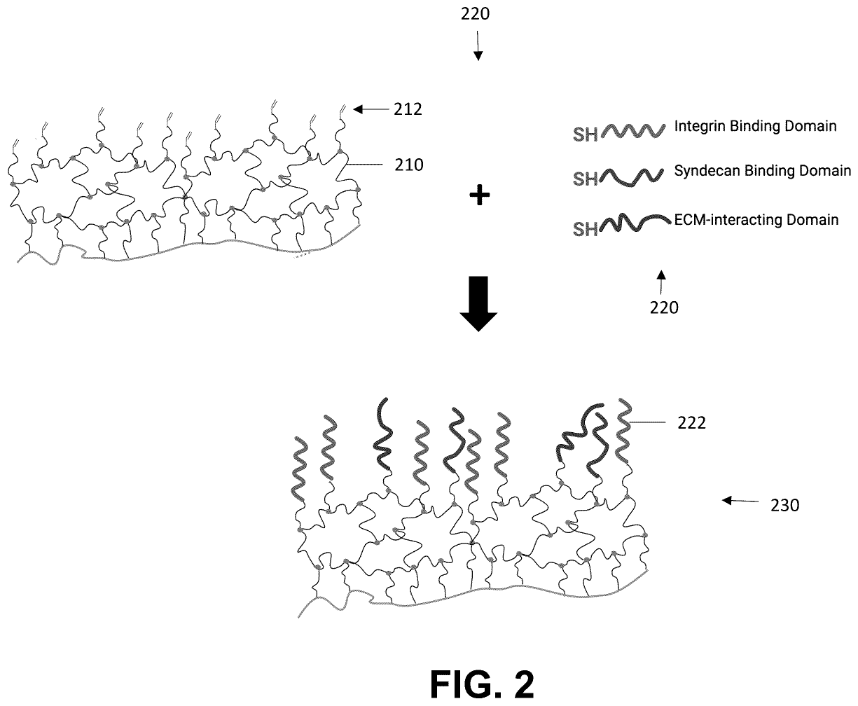

FIG. 2 is a schematic diagram of an embodiment of a reaction scheme 200 used to functionalize the surface of a tissue scaffold. In some embodiments, the reaction scheme 200 may take advantage of the pendant chemical groups resulting from the 3D printing process. In some embodiments, pendant chemical groups can be modified, or other chemical groups can be added to surface 212 to facilitate reaction scheme 200 and surface modification. The reactants in the reaction scheme 200 are chemical groups 212 at the surface of a scaffold 210 and proteins 220 that include a reactive chemical group reactive with the chemical groups 212 on the scaffold 210. In some embodiments, the reactive chemical groups on the proteins 220 may be in terminal positions or near terminal positions. The reaction scheme 200 binds the proteins 220 to the surface of the scaffold 210 via reaction between the chemical groups 212 on the scaffold and the chemical groups on the proteins to form a scaffold surface 230 that is functionalized with peptides 222. The binding may be a covalent bond.

The reactive chemical group on the proteins 220 may be a thiol, an acrylate, a maleimide, a vinyl sulfone, a norbornene, an acrylamide, an acrylonitrile, and/or a methacrylate. The proteins 220 may each include a single reactive chemical group or multiple reactive chemical groups of the same type or different types. If the reactive group is a thiol, the thiol may be naturally present in the protein or synthetically added to the protein to create a thiolated peptide. In some embodiments, the thiol group is part of a cysteine amino acid.

The reaction scheme 200 may be a click chemistry reaction. For example, the chemical group on a protein may be a thiol group, the chemical group at the scaffold surface may be an acrylate, and the reaction may be a thiol-Michael addition click reaction.

The peptides that functionalize the tissue scaffold surface promote cell interaction with the tissue scaffold. The peptides may be selected based on the specific cell types being used and the desired interaction with the surface, e.g., adhesion and growth to confluence. The scaffold surface may be functionalized with different types of peptides to promote different types of cell interactions. Some peptides (e.g., that bind to integrin and/or syndecan) may create focal adhesion points that lead to cell proliferation, some peptides may capture extracellular matrix (ECM) (e.g., fibronectin, laminin, and/or collagen) to create signaling loops that may lead to tissue formation, and some peptides may promote MMP-dependent remodeling. As an example, peptides that capture ECM may include peptides with an affinity for fibronectin, laminin, and/or collagen.

In some embodiments, the peptides may have an affinity for at least one of syndecan, integrin, fibronectin, collagen IV, or laminin. Preferably, the peptides have an affinity for at least two of the above peptides promote more efficient cell attachment. Here, efficient cell attachment relates to the number of cells that attach to the tissue scaffold after a period of time and/or to the shape of the cells attached to the tissue scaffold (e.g., for a certain type of cells, cells with a round shape may be considered not viable and cells with an elongated shape may be considered viable). In some embodiments, the tissue scaffold is functionalized with only a single type of peptides. In other embodiments, the tissue scaffold is functionalized with more than one type of peptide to promote more efficient cell attachment to the tissue scaffold.

As an example, the peptides used for surface modification may include one or more of the thiolated peptides in Table 1.

TABLE 1
Thiolated peptides for surface modification

| Peptide name | Sequence | MW (Da) |
|---|---|---|
| RGDS | CGRDRGDSPY (SEQ ID NO: 1) | 1138 |
| PHSRNKRGD | PHSRNGGGK(GGGERCG)GGRGDSPY (SEQ | 2357 |
| | ID NO: 2) (GGGERCG disclosed as SEQ | |
| | ID NO: 3) | |
| AG73 | GCREKKRKRLQVQLSIRT (SEQ ID NO: 4) | 2241 |
| IKVAV | GCREIKVAV (SEQ ID NO: 5) | 1015 |
| FN-Binder | GCREKKTLQPVYEYMVGV (SEQ ID NO: 6) | 2141 |
| BM-Binder | GCREISAFLGIPFAEPPMGPRRFLPPEPKKP | 3477 |
| | (SEQ ID NO: 7) | |
| MMP-Peptide | GCRDVPMSMRGGDRCG (SEQ ID NO: 11) | 1738 |
| GFOGER | GGYGGGPG(GPP)₅GFOGER(GPP)₅GPC | 3979 |
| | (SEQ ID NO: 8) | |

The peptides listed in Table 1 are chemically defined and reproducible. These peptides can be prepared with high purity (>90%). These peptides include at least one cysteine amino acid (denoted by “C”) that can react with chemical groups on the surface of the tissue scaffold. RGDS (SEQ ID NO: 12) is a fibronectin-derived peptide. PHSRNKRGD (SEQ ID NO: 9) is a fibronectin-derived peptide with a syndecan and integrin binding domain. AG73 is a laminin-derived peptide recognized by syndecans. IKVAV (SEQ ID NO: 13) is a laminin-derived peptide recognized by syndecans. FN-Binder is a peptide with an affinity for fibronectin. BM-Binder is a peptide with an affinity for collagen IV and laminin. MMP-peptide is an MMP-sensitive crosslinker that induces fast degradation. GFOGER (SEQ ID NO: 10) is an integrin-binding peptide derived from Collagen I.

The tissue scaffold may be functionalized in different combinations of peptides. For example, the combination of peptides on the surface may include PHSRNKRGD (SEQ ID NO: 9), AG73, and BM-Binder. Another combination may include PHSRNKRGD (SEQ ID NO: 9), AG73, BM-Binder, and FN-Binder. Another combination may include GFOGER (SEQ ID NO: 10), AG73, and BM-Binder. Another combination may include GFOGER (SEQ ID NO: 10), AG73, BM-Binder, and FN-Binder. Another combination may include PHSRNKRGD (SEQ ID NO: 9), GFOGER (SEQ ID NO: 10), AG73, and BM-Binder. Another combination may include PHSRNKRGD (SEQ ID NO: 9), GFOGER (SEQ ID NO: 10), AG73, BM-Binder, and FN-Binder. When combinations of peptides are used, each peptide may be present in a molar ratio relative to other peptides of about 0 to about 10 (e.g., about 0.5, 1, 2, 3, 4, 5, or 10).

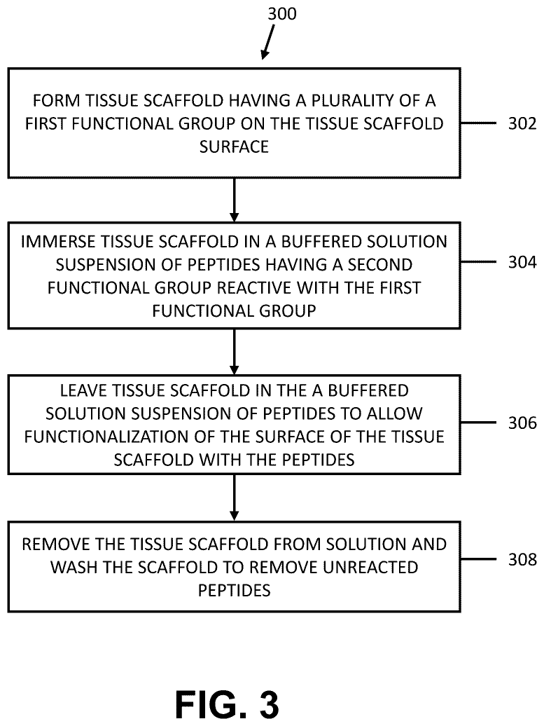

FIG. 3 is a flowchart depicting an embodiment of a method 300 of functionalizing the surface of a tissue scaffold. The method 300 includes a step 302 of forming the tissue scaffold so that it has a plurality of functional chemical groups on its surface that can bind to the peptides of interest.

In step 304, the tissue scaffold is immersed wholly or partially in a buffered solution suspension of the peptides of interest (a single type or a combination of different peptides). The peptides include a second functional chemical group that can react with the functional group on the scaffold surface to bind the peptides to the surface of the scaffold.

The solution suspension of peptides is prepared by suspending the peptides in a buffered solution. In some embodiments, the peptides are suspended in solution from a lyophilized state. The buffered solution can have a pH of about 7.0 to about 10. For example, the buffered solution can have a pH of about 7.5 to about 8.5. The buffered solution may include any suitable buffer, including HEPES (N-2-hydroxyethylpiperazine-N′-2-ethanesulfonic acid), PBS (phosphate-buffered saline), sodium borate, or any combination thereof. In one example, the buffered solution includes both HEPES and PBS. The buffer solution is preferably sterile. Once this solution is created, it may be used immediately or stored in sterile conditions until ready to use.

The concentration of peptides in the suspended solution can be about 0.5 millimolar (mM) to about 20 mM. Table 2 includes some example peptide formulations that may be used to functionalize the surface of the scaffold.

TABLE 2
Combination peptide formulations

| Peptide formulation | Total concentration | | Concentration (mM) |
|---|---|---|---|
| P/A/B | 3 | mM | 2/.5/.5/ |
| P/A/B/F | 3.5 | mM | 2/.5/.5/.5 |
| P/A/B/F | 4.5 | mM | 2/.5/1/1 |
| G/A/B | 3 | mM | 2/.5/.5/ |
| G/A/B/F | 3.5 | mM | 2/.5/.5/.5 |
| G/A/B/F | 4.5 | mM | 2/.5/1/1 |
| P/G/A/B | 5 | mM | 2/2/.5/.5 |
| P/G/A/B/F | 5.5 | mM | 2/2/.5/.5/.5 |
| P/G/A/B/F | 6.5 | mM | 2/2/.5/1/1 |

In Table 2, P is PSHRNKRGDS (SEQ ID NO: 14), B is BM-Binder, A is AG73, F is FN-Binder, and G is GFOGER (SEQ ID NO: 10). Column 2 of Table 2 lists the total peptide concentration in solution used to modify the surface of a tissue scaffold. Column 3 of Table 2 includes the concentrations of individual peptides in the solution, where concentrations are listed in the same order as in column 1, and the sum of the individual concentrations is the total concentration in column 2.

In step 306, the tissue scaffold is soaked in the solution suspension of peptides for a period of time sufficient to allow reaction between the peptides and the surface of the scaffold. The tissue scaffold may be left in the solution for any amount of time suitable for surface modification. In some embodiments, the amount of time is about 1 hour to about 18 hours (e.g., 1 hour, 2 hours, 5 hours, 10 hours, or 15 hours). The surface concentration of peptides after the tissue scaffold is functionalized is about 0.02 μmol/cm² to about 10 μmol/cm². The temperature of the solution suspension of peptides during step 306 is about 15° C. to about 40° C. For example, the temperature of the solution suspension may be about 20° C. to about 38° C., about 20° to about 27° C., or about 35° C. to about 37° C. In some embodiments the concentration of peptides bonded to the tissue scaffold surface after the tissue scaffold is functionalized is about 0.2-8 μmol/cm², about 0.2-4 μmol/cm², about 3-7 μmol/cm², about 4-6 μmol/cm², about 4 μmol/cm², or about 0.2 μmol/cm². In some embodiments the concentration of peptides after the tissue scaffold is functionalized is at least 0.2 μmol/cm² to ensure sufficient coverage of the majority of the functional groups on the surface of the tissue scaffold.

In step 308, the tissue scaffold is removed from the solution suspension of peptides and washed to remove peptides from the solution that have not reacted with the surface of the scaffold. In some embodiments, the tissue scaffold is not washed to remove peptides that have not reacted with the surface of the scaffold. The tissue scaffold may be washed with a buffer solution (e.g., HEPES, PBS, or a combination thereof) or any other suitable solution. As an example, washing may include soaking the tissue scaffold in buffer or rinsing the tissue scaffold with buffer. As another example, washing may include perfusing the tissue scaffold with buffer for about 8 hours to about 24 hours. Perfusion washing may be preferred when the tissue scaffold has a complex internal structure. In some embodiments, where the tissue scaffold has a complex shape with many channels, the method 300 may be repeated to one to twenty times to increase the amount of peptide bound to the surface of the scaffold.

After step 308, the scaffold may be used right away, where the scaffold is preconditioned with cell media and seeded with cells, or the scaffold may be stored in sterile conditions until needed. The surface-functionalized tissue scaffold is stable and can be stored in sterile conditions for an extended period of time. For example, the surface-functionalized tissue scaffold may be stored for up to about 1 year, 6 months, 1 month, 4 weeks, or 3 weeks.

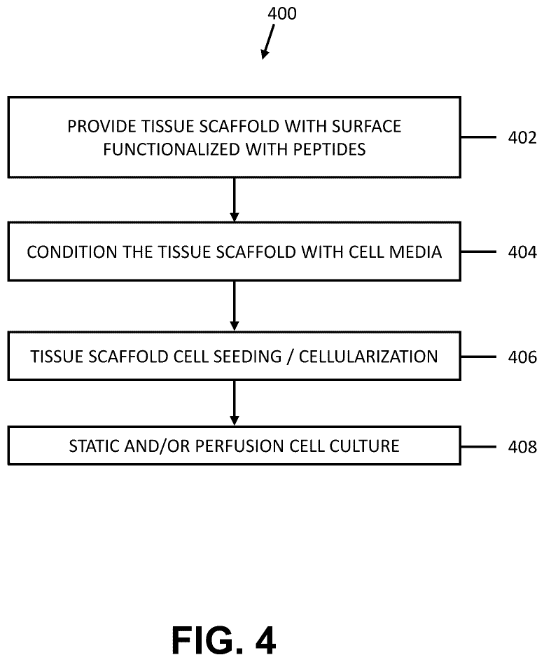

FIG. 4 is a flowchart depicting an embodiment of a method 400 of culturing biological cells on a surface-functionalized tissue scaffold. In the first step 402, the surface-functionalized tissue scaffold is provided. For example, the surface-functionalized tissue scaffold can be prepared according to the method 300 described above and in FIG. 3.

In step 404, the tissue scaffold is preconditioned with cell media. The tissue scaffold is preconditioned by soaking the tissue scaffold in cell media. In some embodiments, preconditioning also includes perfusion of the lumens in the tissue scaffold with cell media in addition to soaking in cell media. The addition of perfusion is particularly useful when preconditioning larger tissue scaffolds. As an example, preconditioning may include soaking the tissue scaffold in static media. As another example, washing may include perfusing the tissue scaffold with media. The scaffold may be preconditioned with media for about 3 hours to about 50 hours.

In step 406, the tissue scaffold is seeded with biological cells. The tissue scaffold may be seeded with about 50,000 cells per cm² to about 500,000 cells per cm² of scaffold area. The cell seeded step may be repeated one to about 10 times to increase the number of cells retained on the scaffold. The seeding process may use static seeding, perfusion seeding, or a combination thereof. As an example, each seeding may be about 1 hour to about 5 hours.

In step 408, the cells seeded on the tissue scaffold are cultured. Culturing may include immersing the tissue scaffold fully or partially in static cell media, perfusing media through the tissue scaffold, or a combination thereof. Perfusion may be intermittent (e.g., 1 minute perfusion followed by 30 minutes static) or continuous. Perfusion may use a flow rate of about 1 mL per minute to about 10 mL per minute (e.g., 6 mL/min). The steps 406 and 408 of cell seeding and culture may be repeated to increase cell attachment and distribution on the tissue scaffold.

As an example, the scaffold may be initially seeded for 3 hours in static media, followed by four rounds of cell seeding and intermittent culture with intermittent perfusion for about two to about 5 days, followed by continuous perfusion.

The biological cells seeded onto the scaffold may be any type of cell useful for formation of the tissue of interest. For example, if the scaffold is intended to be used as a lung scaffold, the biological cells may be primary adult lung cells, including pulmonary artery endothelial cells (PAECs) or small airway epithelial cells (SAECs), or may be induced pluripotent stem cell-derived endothelial cells. These surface-functionalized scaffolds may be used to form functional multicellular organs, such as lungs.

### EXAMPLES

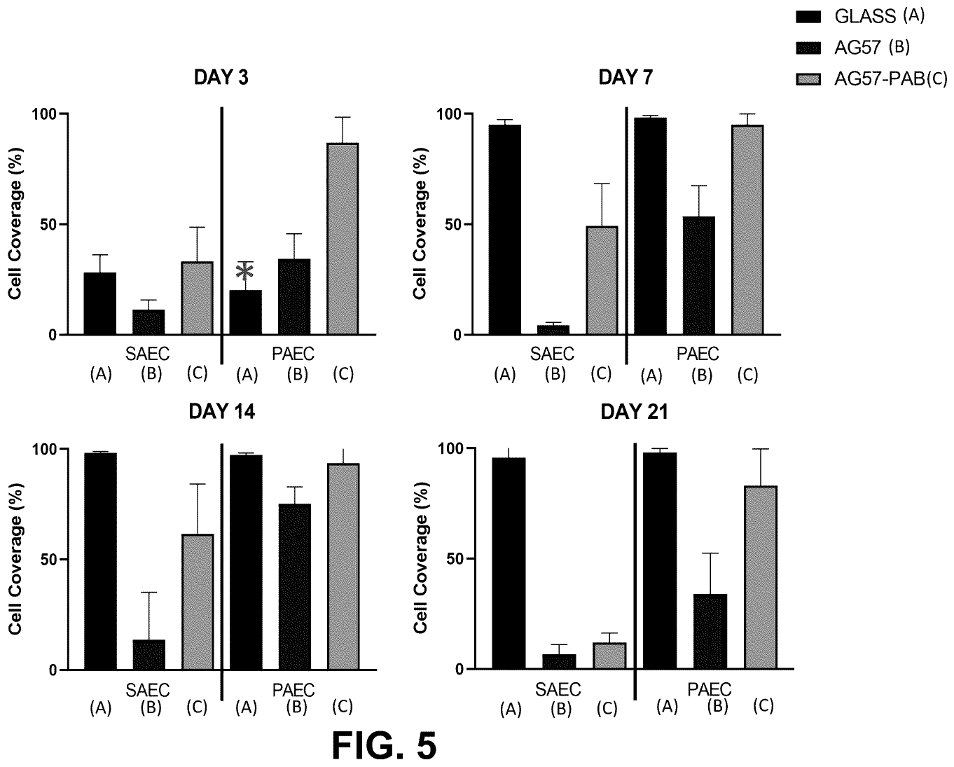

FIG. 5 shows four graphs comparing cell coverage over time for different surfaces. The graphs compare a surface functionalized with peptides to control surfaces. The control surfaces are 1) a bioink used for printing tissue scaffolds without surface functionalization (AG57) and 2) glass. Two types of biological cells were compared—PAECs and SAECs. Cell attachment and proliferation on different surfaces were monitored for 21 days at day 3, 7, 14, and 21. The surface-functionalized scaffold (AG57-PAB, right bars) was functionalized with PHSRNKRGD (SEQ ID NO: 9), AG73, and BM-Binder in a molar ratio of 2/0.5/0.5. The control conditions were the same type of scaffold without functionalization (AG57, middle bars), and a glass surface (left bars). Cell coverage of PAECs on the surface-functionalized scaffold was similar to the glass surface condition even after 21 days of cell culture. In all conditions, cell coverage was greater on the surface-functionalized scaffold than on the scaffold without functionalization.

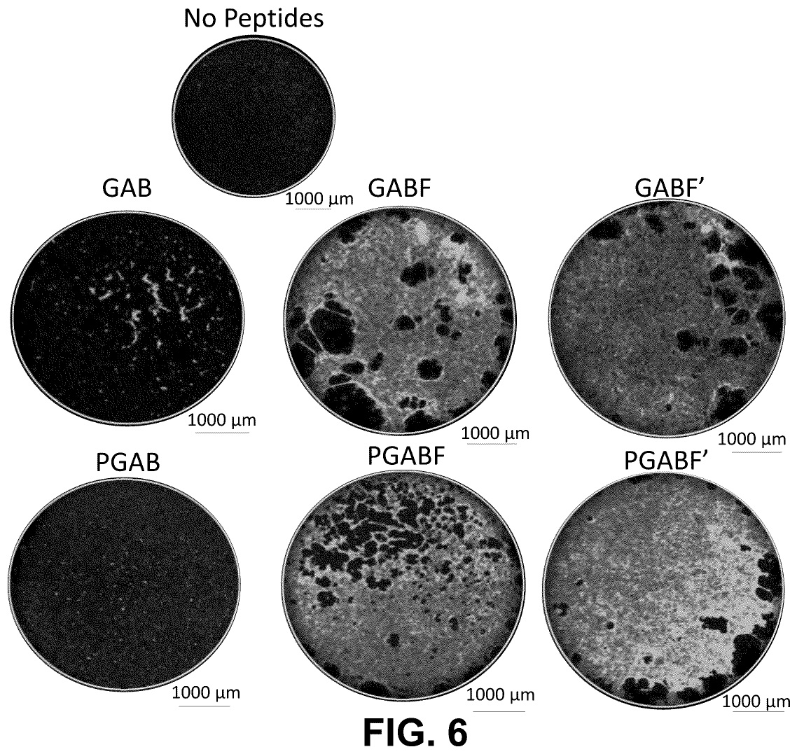

FIG. 6 shows stained disk scans of SAEC attachment and proliferation at day 11 on bioink used for printing tissue scaffolds that was surface-modified with different combinations of peptides prior to cellulation. Bioink surfaces were modified with different cocktails of peptides. The peptide cocktails were G/A/B, G/A/B/F, G/A/B/F′, P/G/A/B, P/G/A/B/F, and P/G/A/B/F′, where P represents PHSRNKRGD (SEQ ID NO: 9) peptide, G represents GFOGER (SEQ ID NO: 10) peptide, A represents AG73 peptide, B represents BM-Binder peptide, and F represents FN-Binder peptide. Peptide concentrations for modification are listed in Table 3. The scan represents about 10 mm diameter of culture surface.

TABLE 3
Combination Peptide Formulations

| Peptide | Concentration (mM) |
|---|---|
| G/A/B | 2/0.5/0.5 |
| G/A/B/F | 2/0.5/0.5/0.5 |
| G/A/B/F′ | 2/0.5/1/1 |
| P/G/A/B | 2/2/0.5/0.5 |
| P/G/A/B/F | 2/2/0.5/0.5/0.5 |
| P/G/A/B/F′ | 2/2/0.5/1/1 |

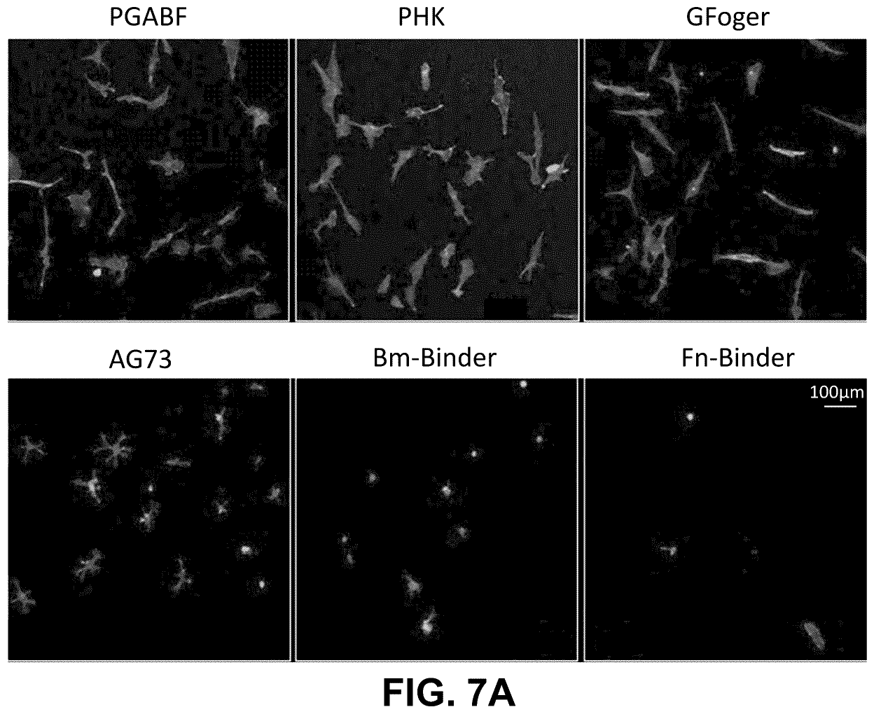

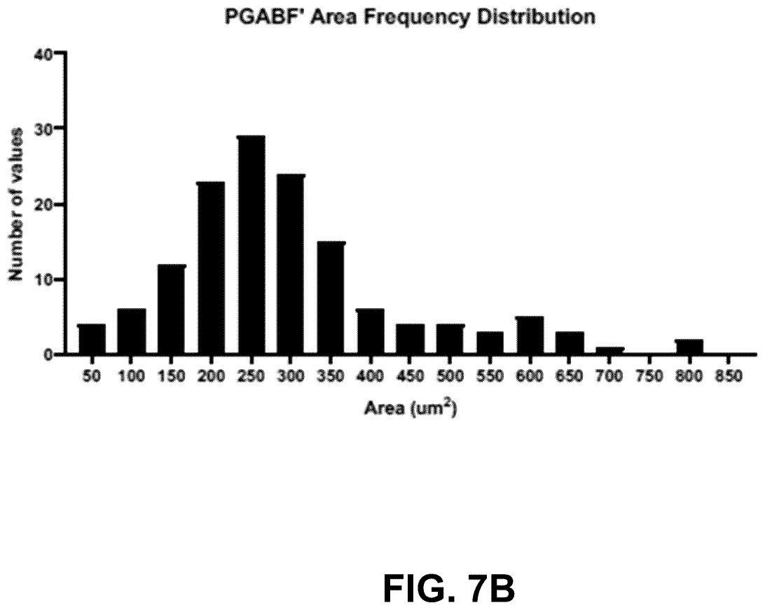

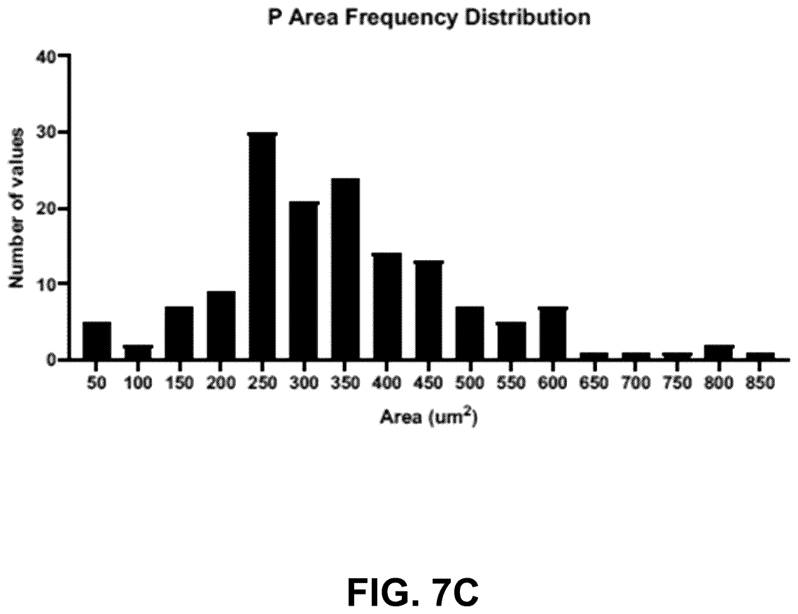

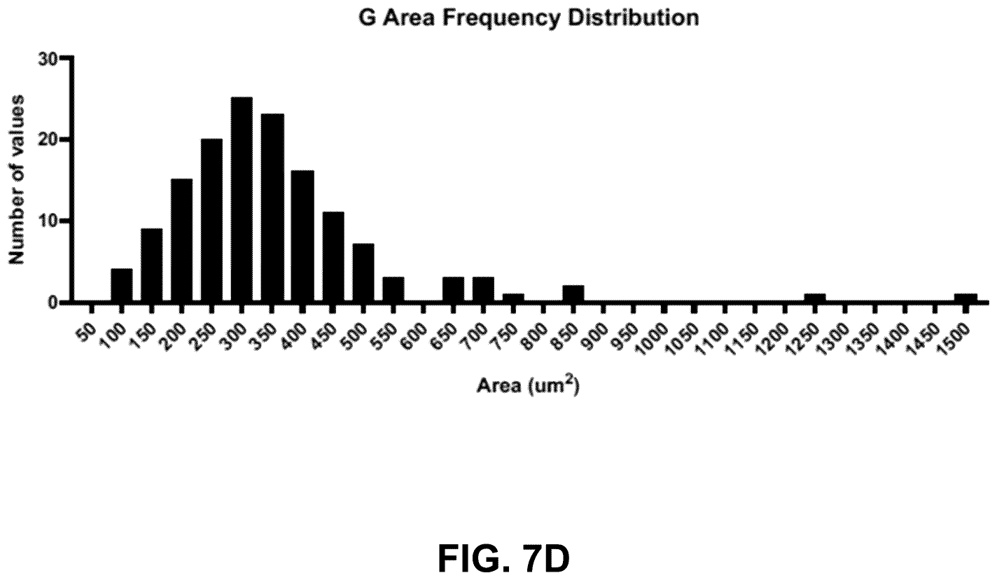

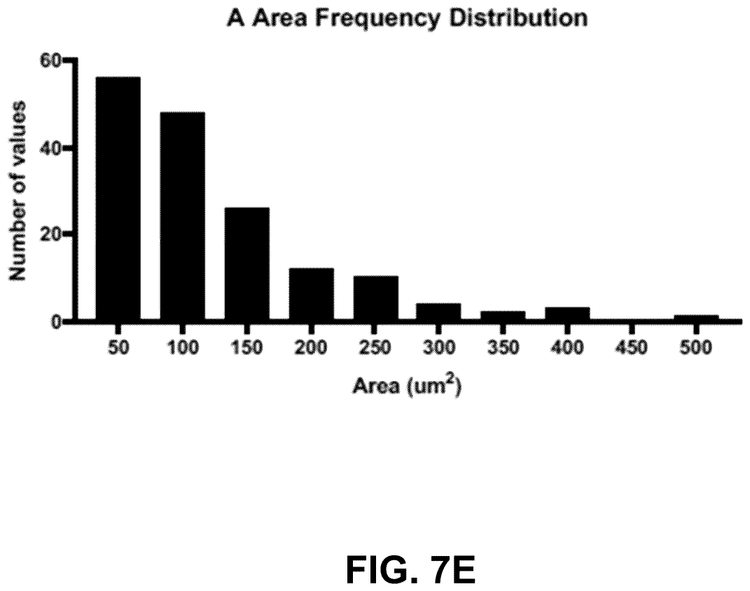

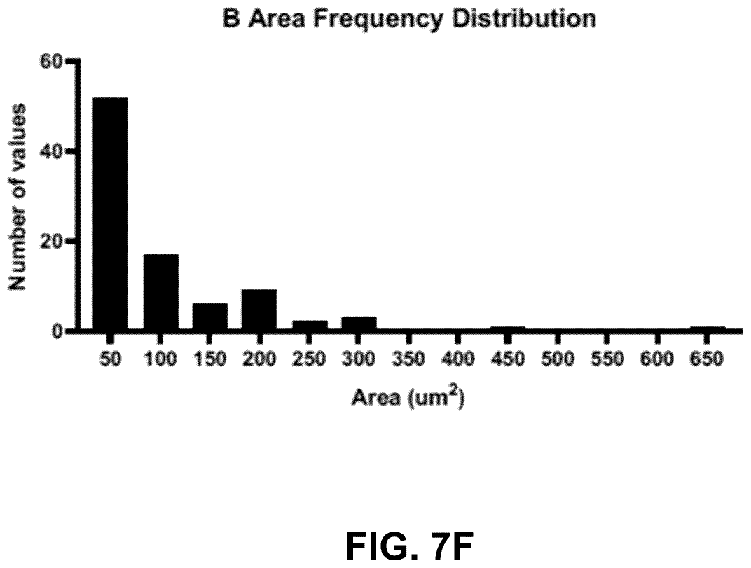

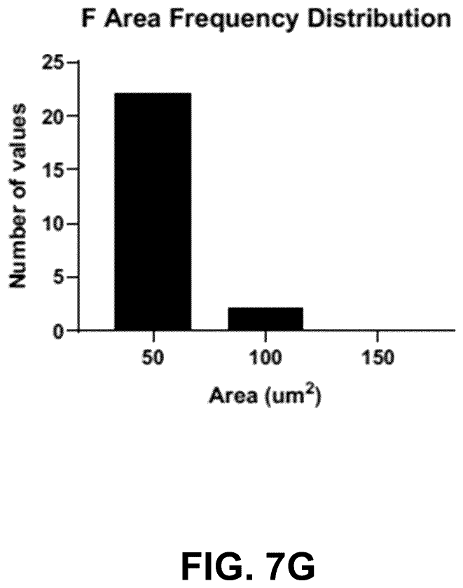

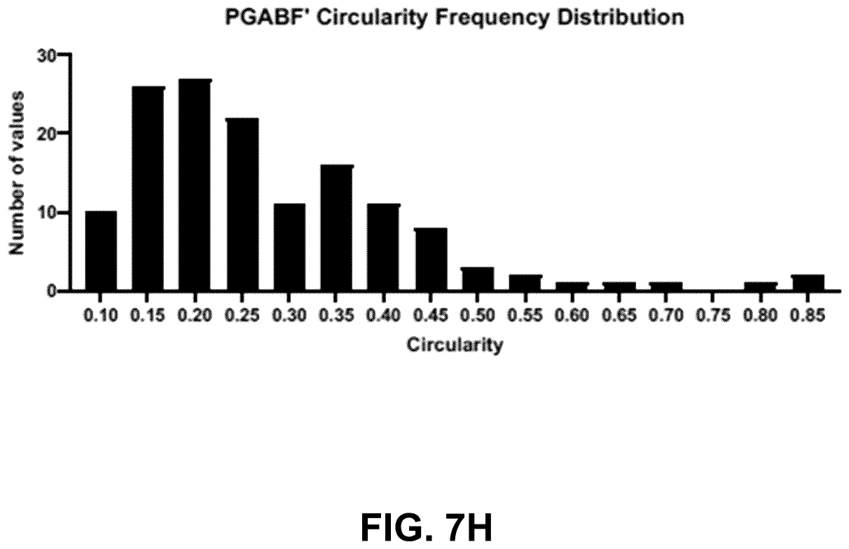

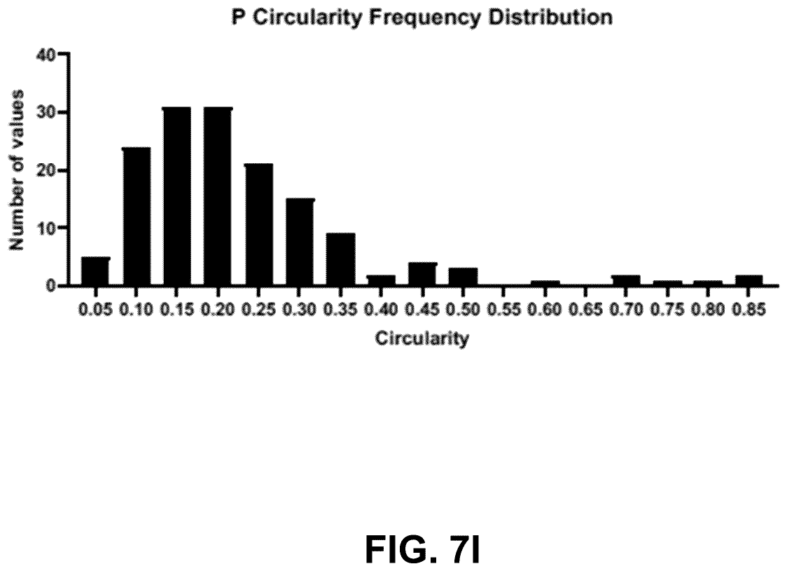

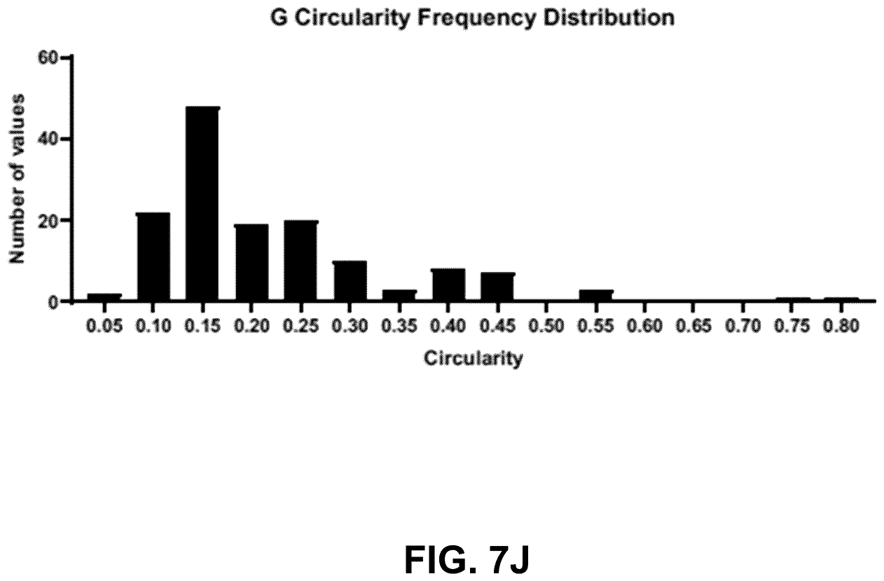

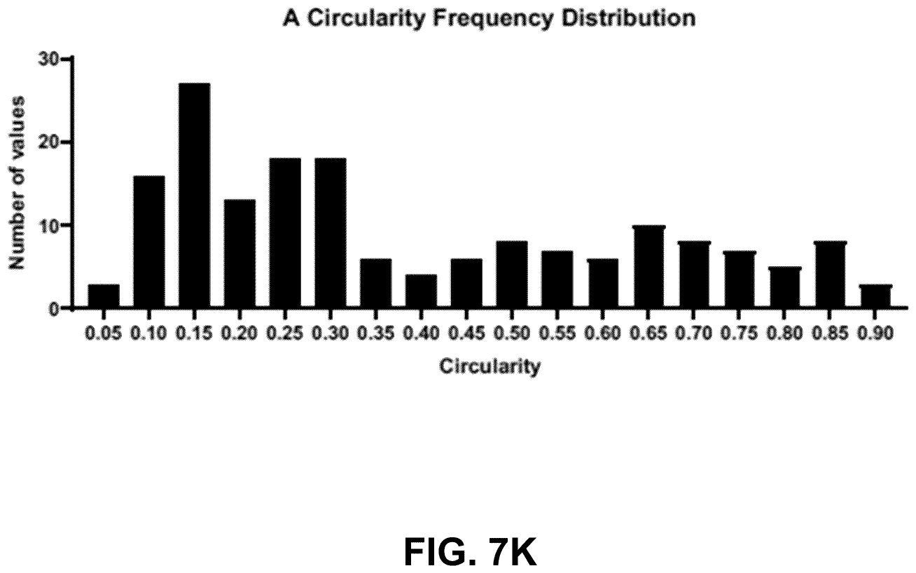

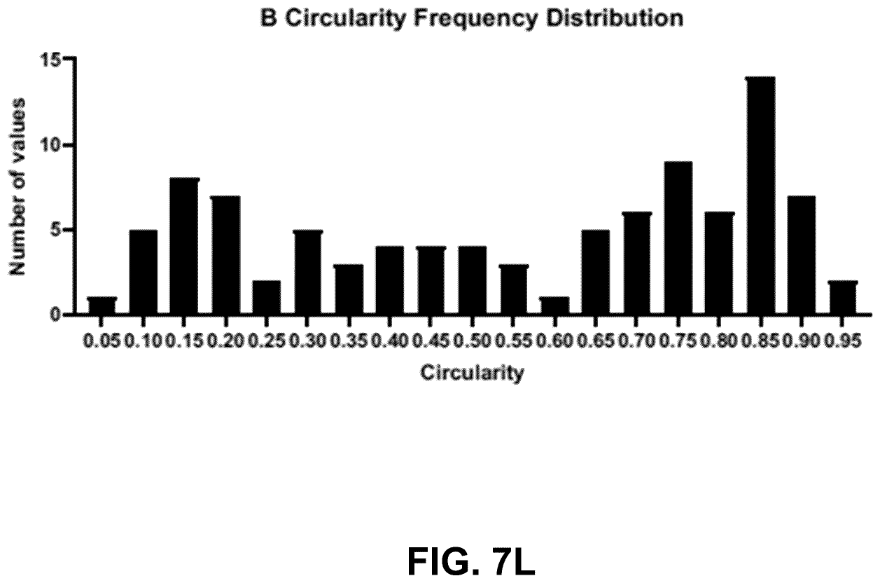

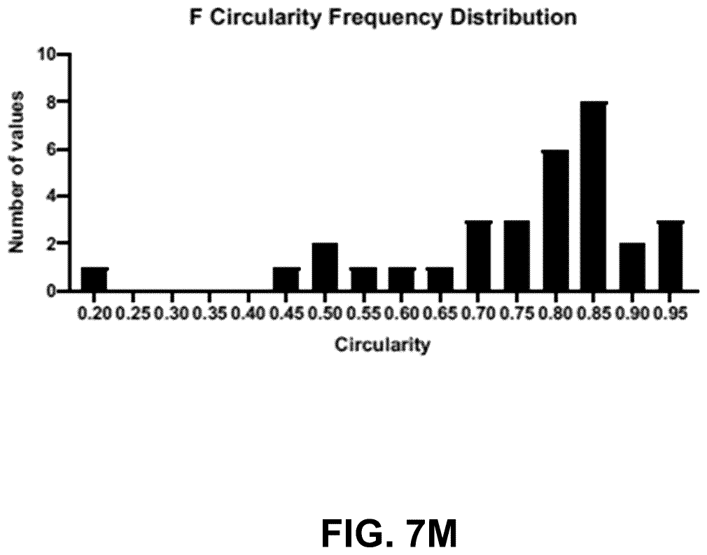

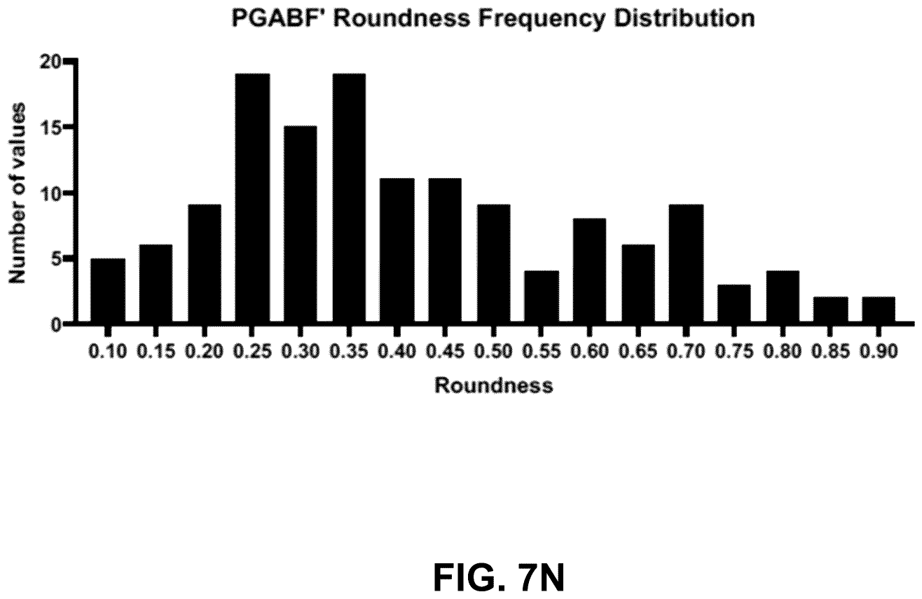

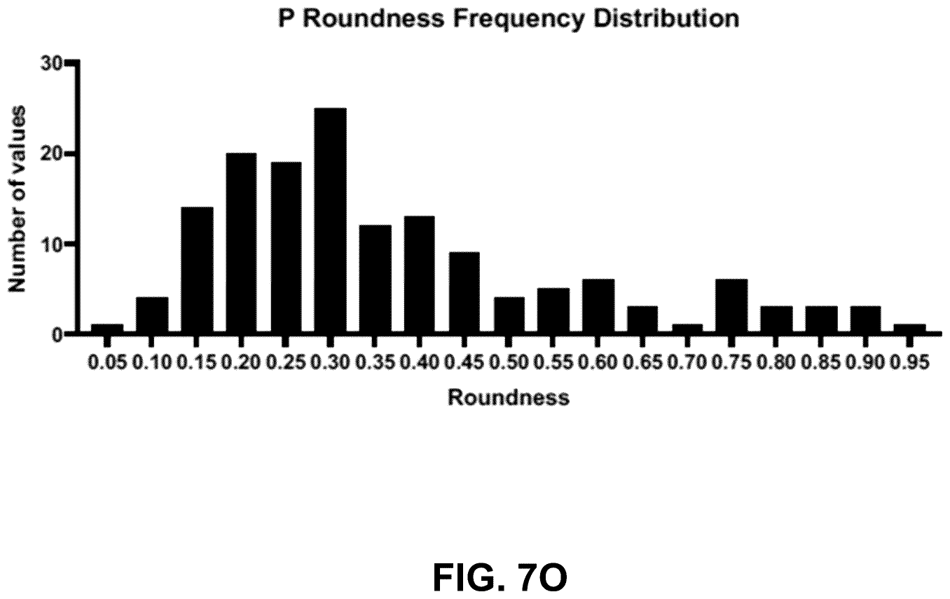

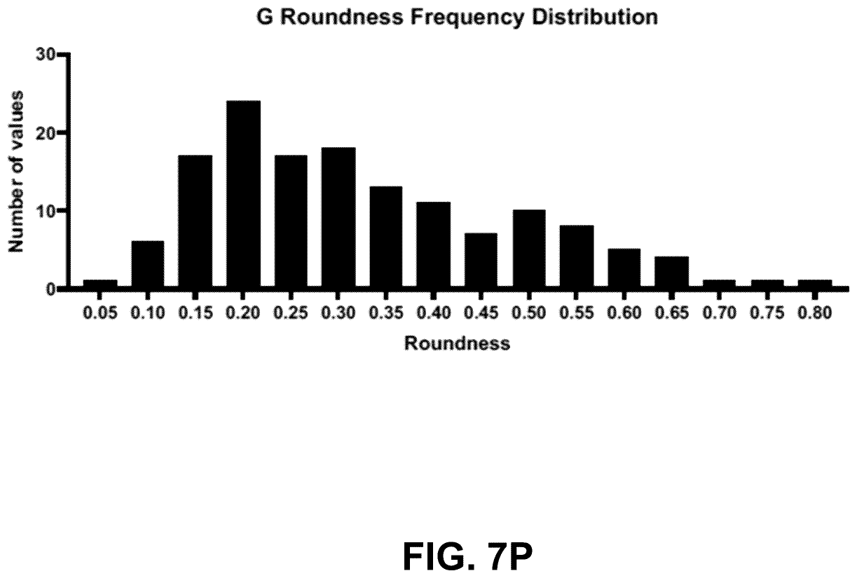

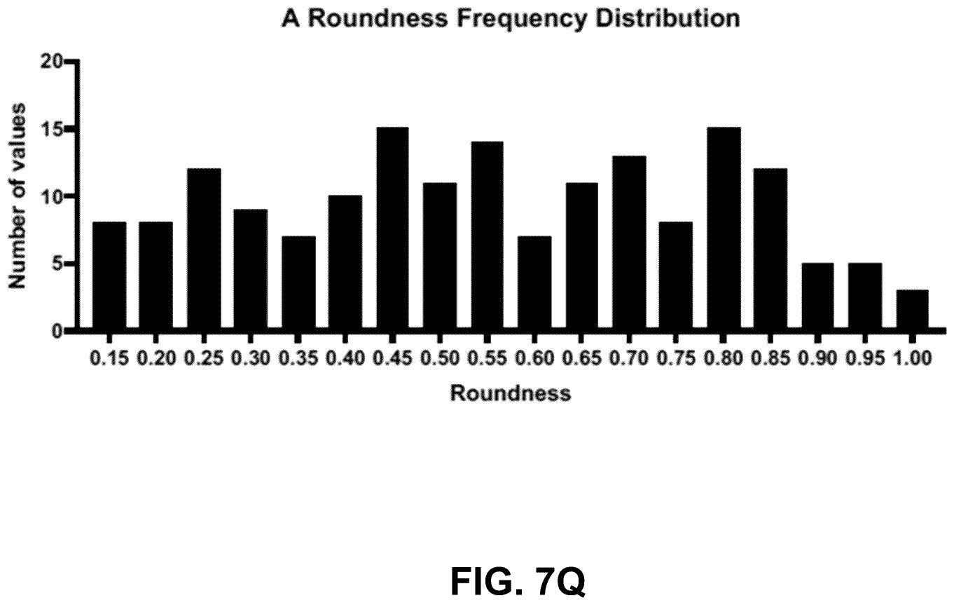

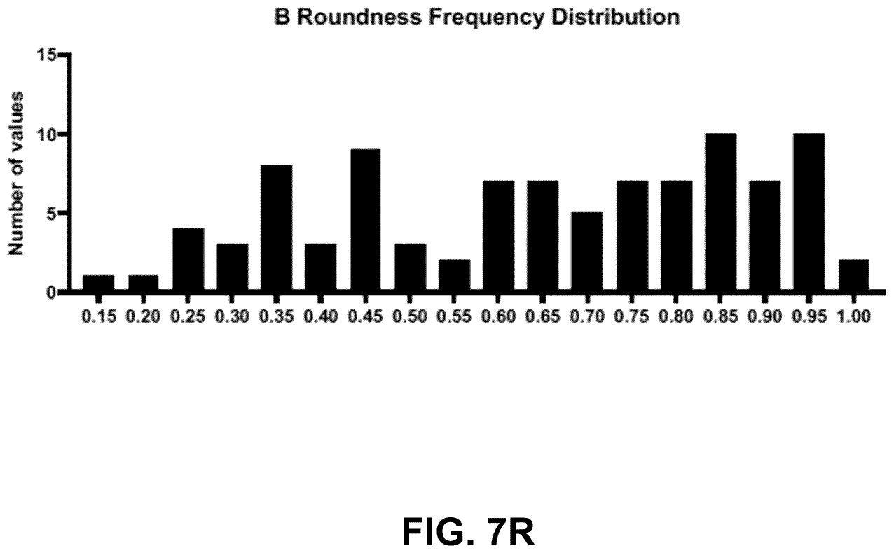

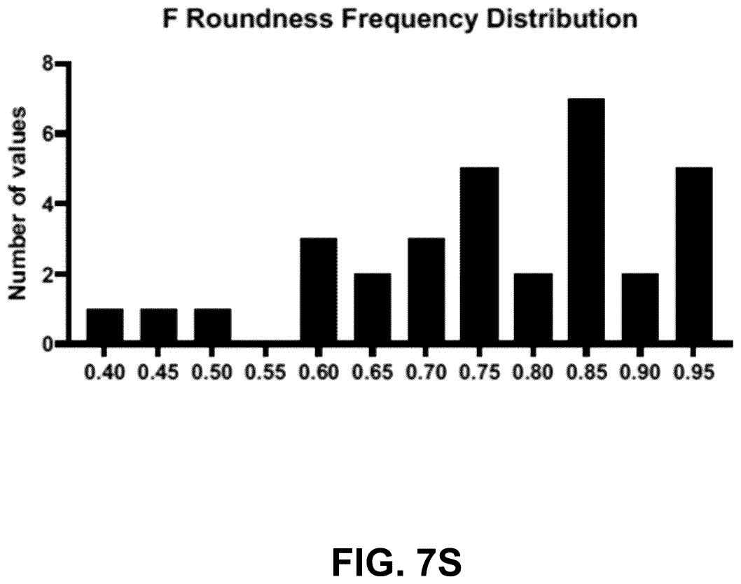

FIGS. 7A-7S illustrate PAEC morphology after 24 hours on discs surface modified with different peptides at a concentration of 2 μmol/cm². FIG. 7A are stained disk scans of PAECs cultured on 6 different surface modification peptides after 24 hours. FIGS. 7B-7S are graphs of FIJI analysis of circularity and roundness frequency distribution of the PAECs cultured on 6 different surface modification peptides after 24 hours.

For the experiments in FIGS. 7A-7S, 18 multi-wells were washed in 70% RA. The multi-wells were split into 6 groups of surface modification, which were P/G/A/B/F, PHSRNKRGD (SEQ ID NO: 9) (referred to as “PHK” in the figures), AG73, GFOGER (SEQ ID NO: 10), BM-Binder, and FN-Binder. Surface modification was conducted with the peptide solutions in Table 4. Peptides were prepared at 2 μmol/cm² concentrations for surface modification, but the actual concentration was adjusted for the thiol concentration. Following surface modification, wells were seeded with PAECs at a concentration of 625000 per cm². 24 hours after seeding, the wells were fix stained and imaged to quantify cell morphology. Model surface area was 10.1 cm².

TABLE 4
Peptide Solutions for Surface Modification for FIGS. 7A-7S.

| | | | | GFOGER | | |
|---|---|---|---|---|---|---|
| | | | | (SEQ ID | BM- | FN- |
| | P/G/A/B/F′ | P | AG73 | NO: 10) | Binder | Binder |
| Component | (μL) | (μL) | (μL) | (μL) | (μL) | (μL) |
| PHK | 155 | 504 | — | — | — | — |
| GFOGER | 155 | | — | 504 | — | — |
| (SEQ ID | | | | | | |
| NO: 10) | | | | | | |
| AG73 | 78 | — | 1008 | — | — | — |
| BM-Binder | 155 | — | — | — | 1008 | — |
| FN-Binder | 310 | — | — | — | — | 1800 |
| 10X | 200 | 200 | 200 | 200 | 200 | 200 |
| HEPES | | | | | | |
| DI Water | 947 | 1296 | 792 | 1296 | 792 | — |
| Actual | 1.69 | 1.55 | 1.29 | 1.75 | 1.68 | 1.85 |
| Conc. | μmol/cm² | μmol/cm² | μmol/cm² | μmol/cm² | μmol/cm² | μmol/cm² |

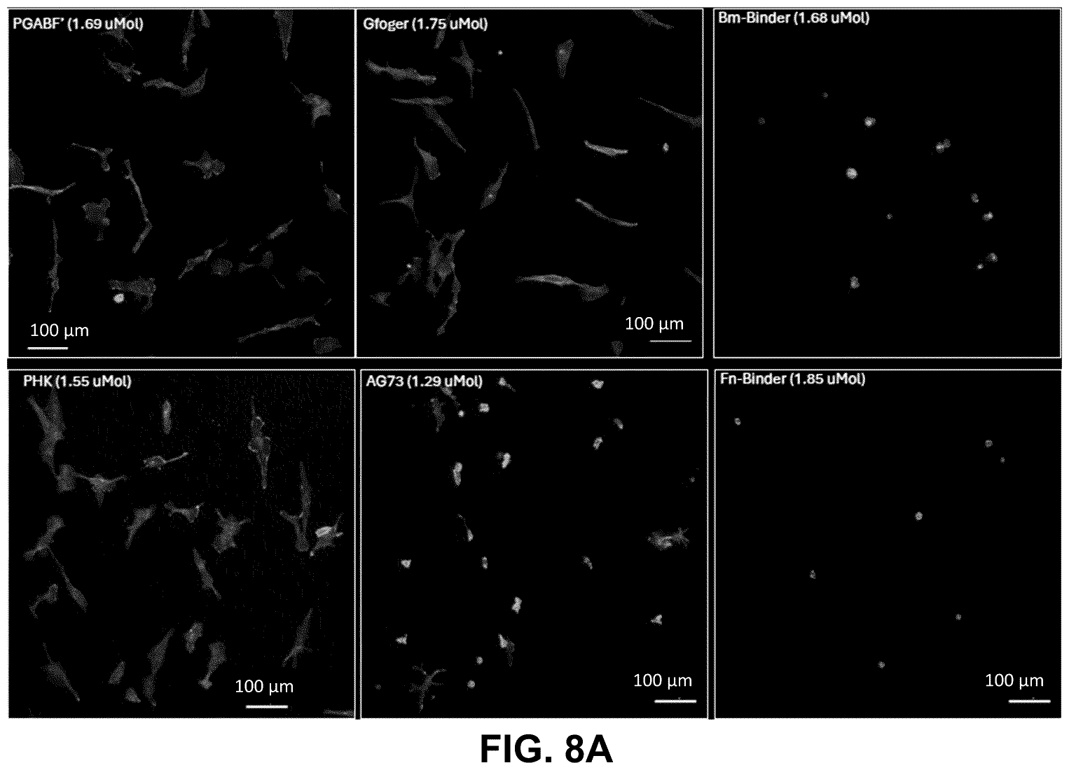

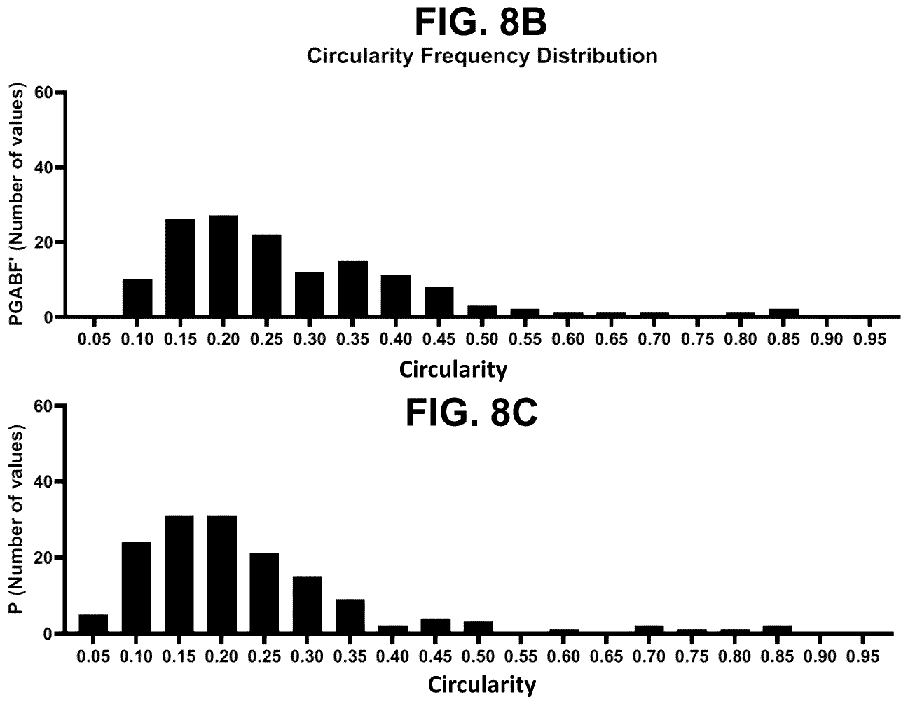

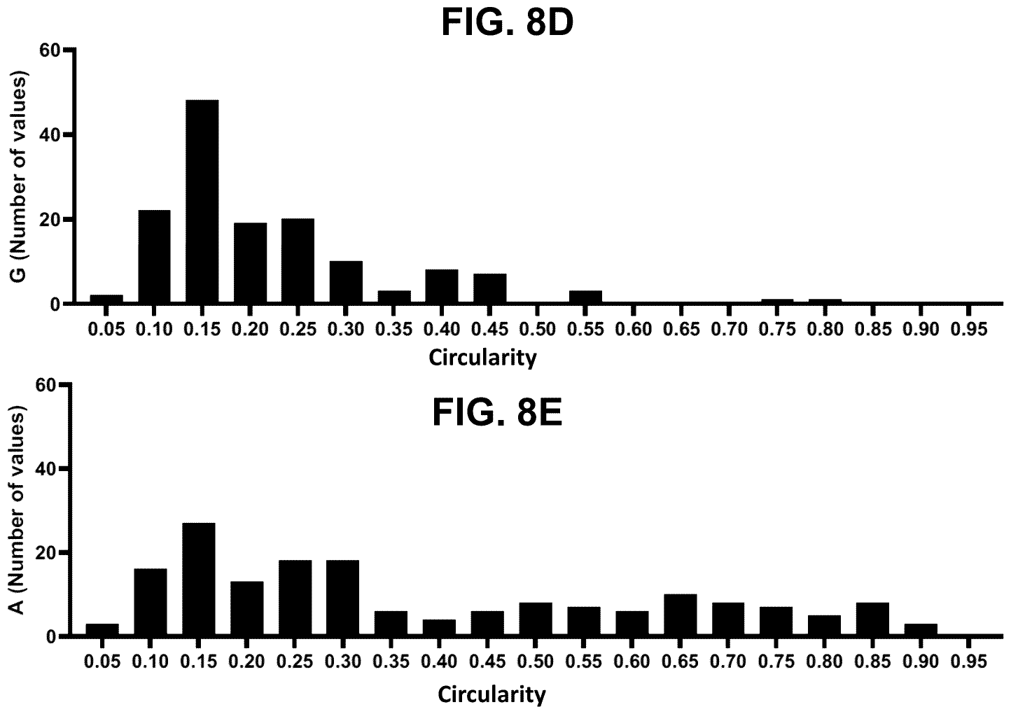

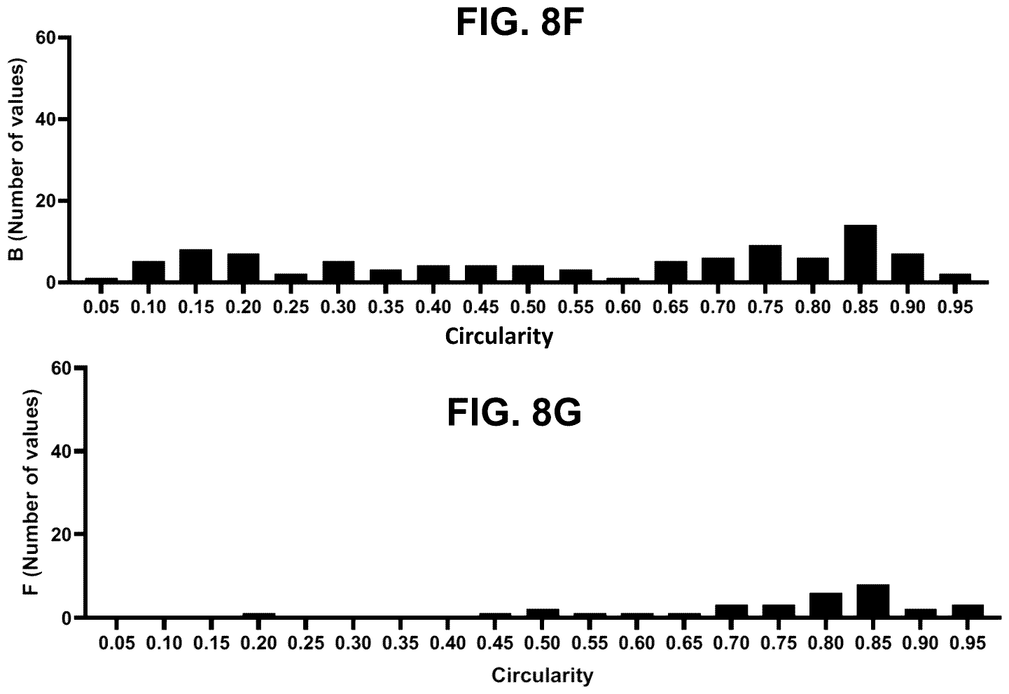

FIGS. 8A-10G provide data on experiments to compare cell seeding and proliferation on surfaces with single peptide or combination peptide modification at different concentrations, 2 μmol/cm² and 0.2 μmol/cm². The solutions used for peptide modification had the actual peptide concentrations noted in Table 5.

TABLE 5
Actual and Theoretical Peptide Concentrations
in Tests 1-3 (μmol/cm²)

| | | | | | GFOGER | | |
|---|---|---|---|---|---|---|---|
| | | P/G/A/ | | | (SEQ ID | BM- | FN- |
| Test | Conc. | B/F′ | P | AG73 | NO: 10) | Binder | Binder |
| 1 | Theor. | 2 | 2 | 2 | 2 | 2 | 2 |
| | Actual | 1.69 | 1.55 | 1.29 | 1.75 | 1.68 | 1.85 |
| 2 | Theor. | 2 | 0.62 | 0.16 | 0.62 | 0.3 | 0.3 |
| | Actual | 1.69 | 0.48 | 0.1 | 0.54 | 0.25 | 0.28 |
| 3 | Theor. | 0.2 | 0.062 | 0.016 | 0.062 | 0.03 | 0.03 |
| | Actual | 0.17 | 0.048 | 0.01 | 0.054 | 0.025 | 0.028 |

FIGS. 8A-8G illustrate PAEC morphology after 24 hours on discs surface modified with various peptides at 2 μmol/cm² concentration according to Test 1. FIG. 8A are stained disk scans of PAECs cultured on 6 different surface modification peptides. FIGS. 8B-8G are graphs of FIJI analysis of circularity frequency distribution.

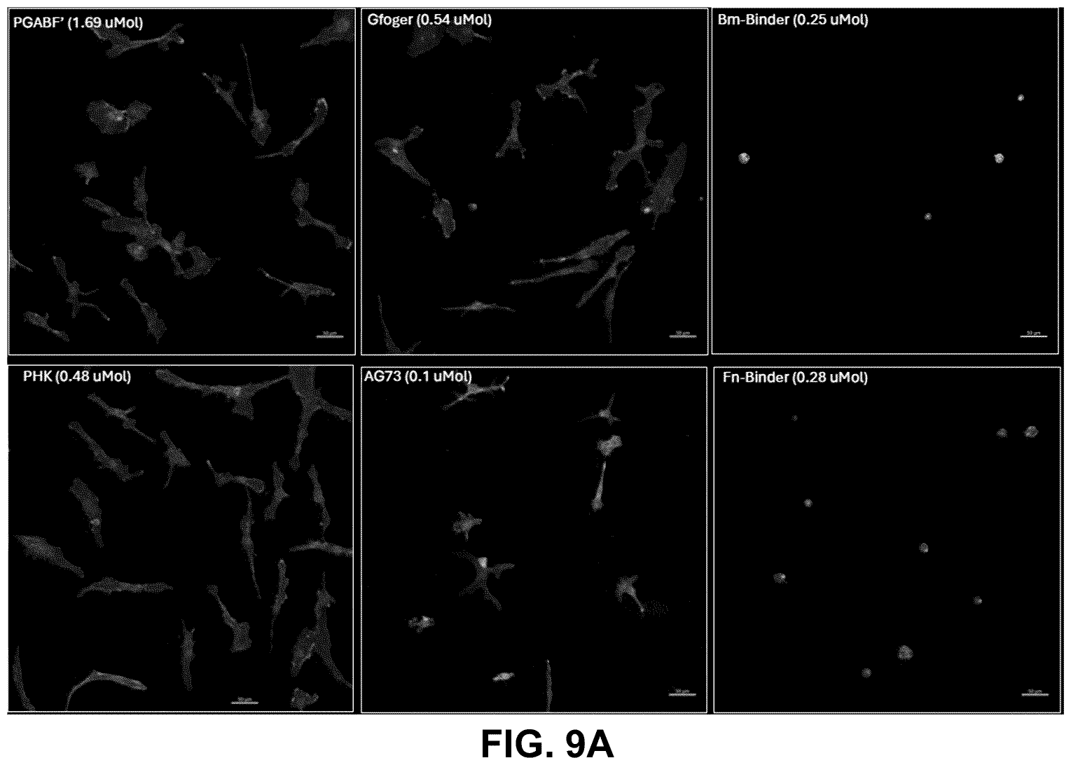

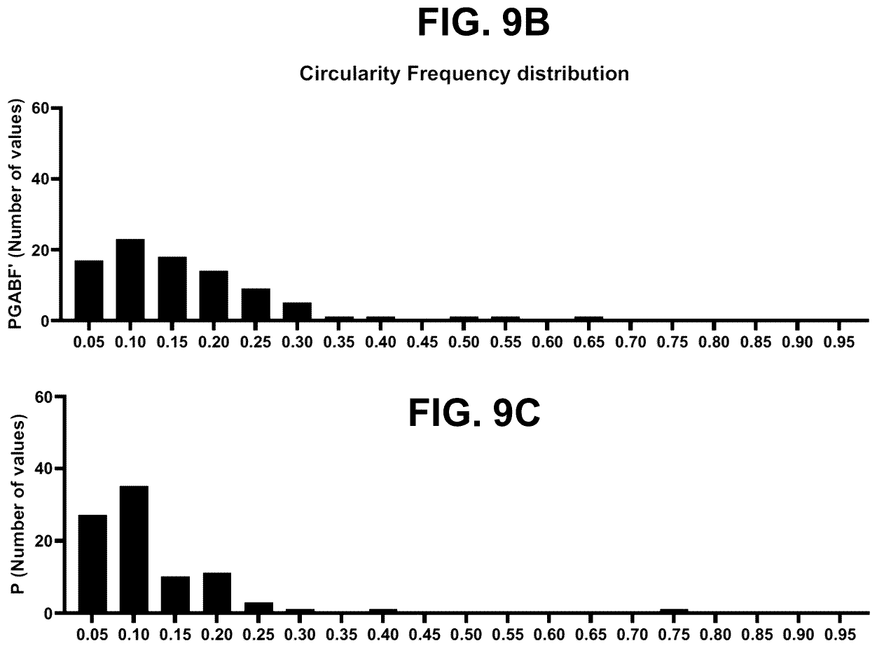

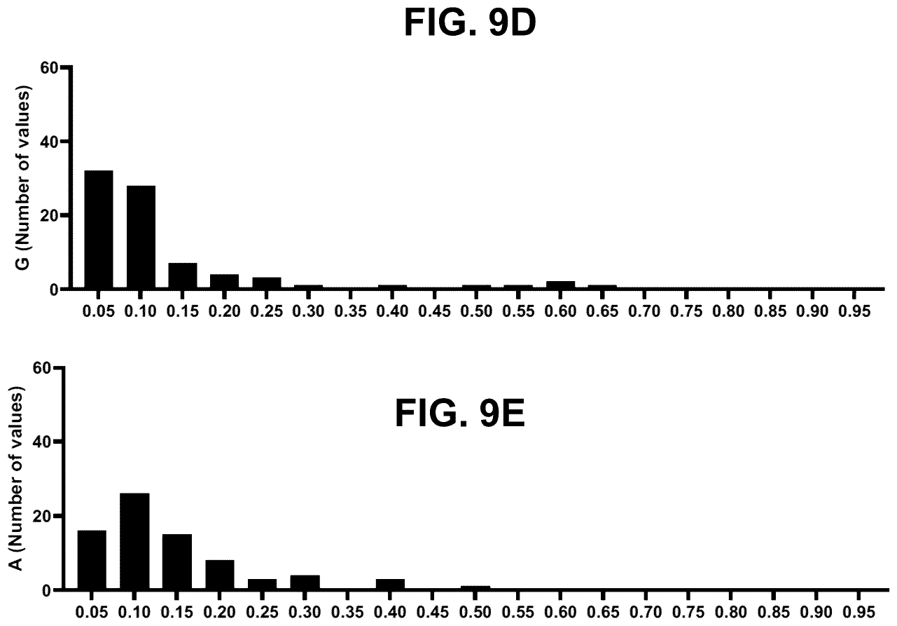

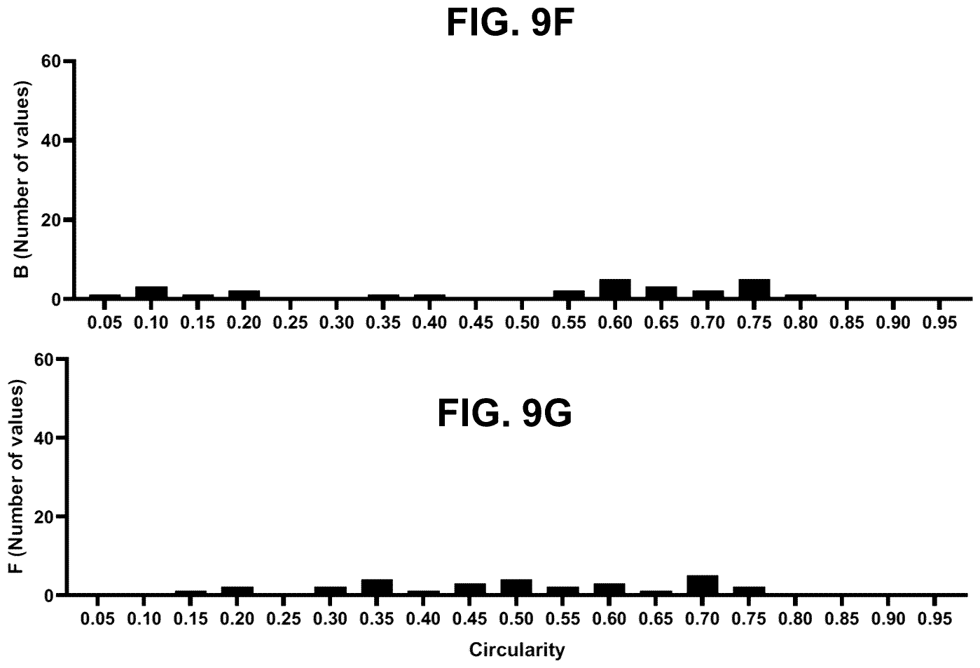

FIGS. 9A-9G illustrate PAEC morphology after 24 hours on discs surface modified with various peptides at 2 μmol/cm² concentration adjusted to the volume used in the PGABF′ condition according to Test 2. FIG. 9A are stained disk scans of PAECs cultured on 6 different surface modification peptides. FIGS. 9B-9G are graphs of FIJI analysis of circularity frequency distribution.

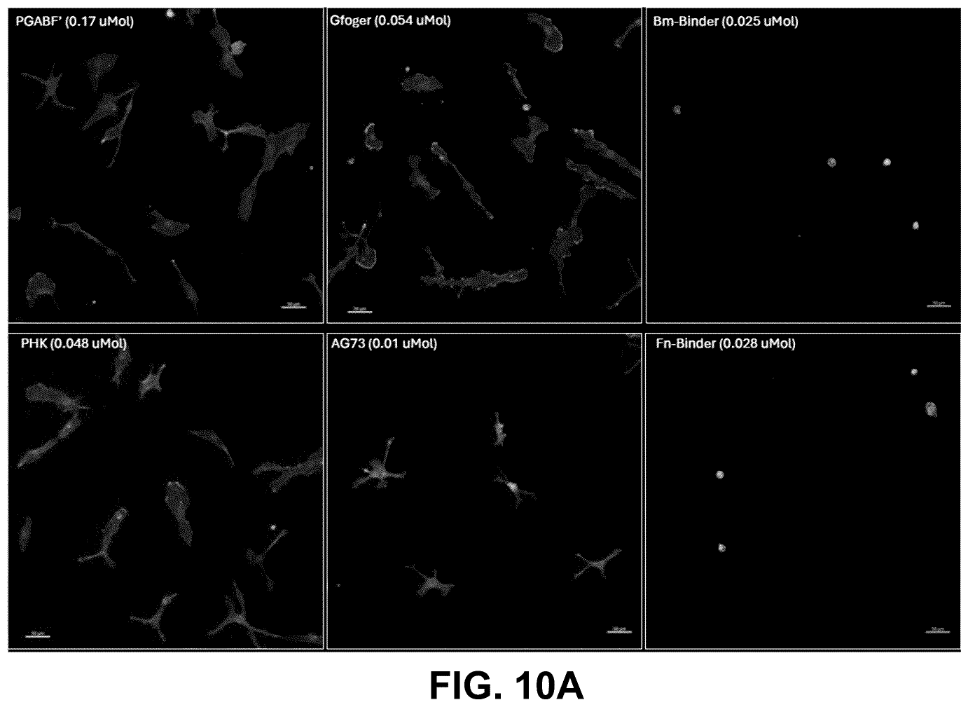

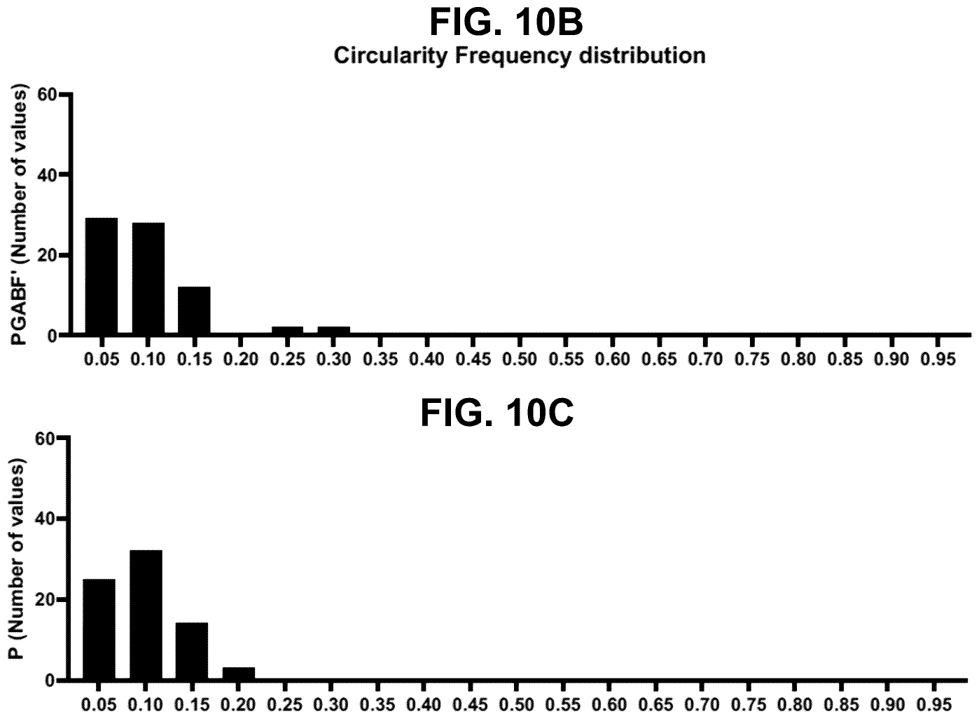

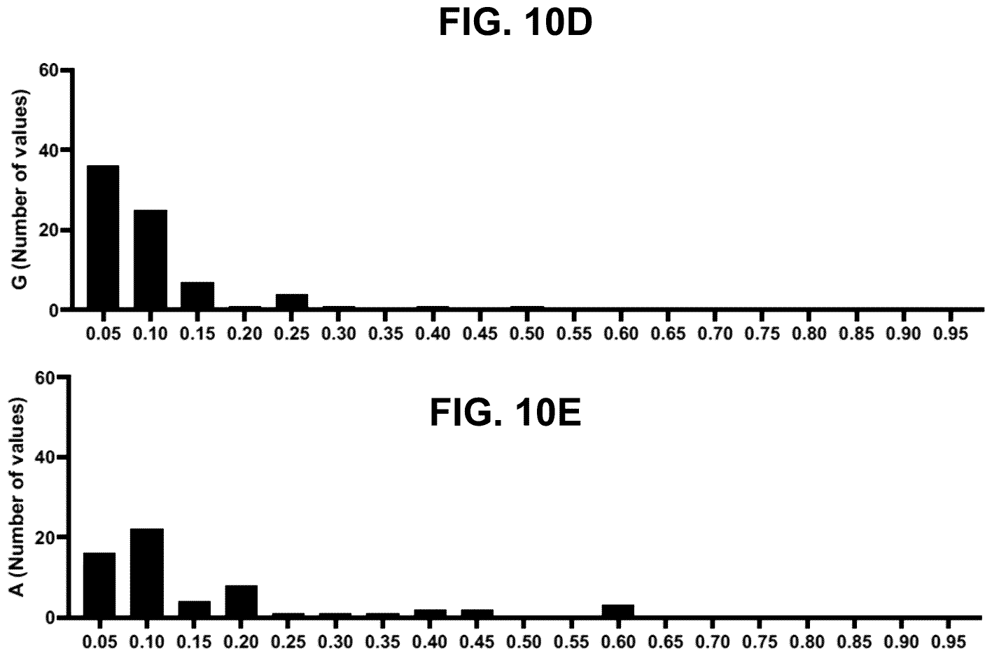

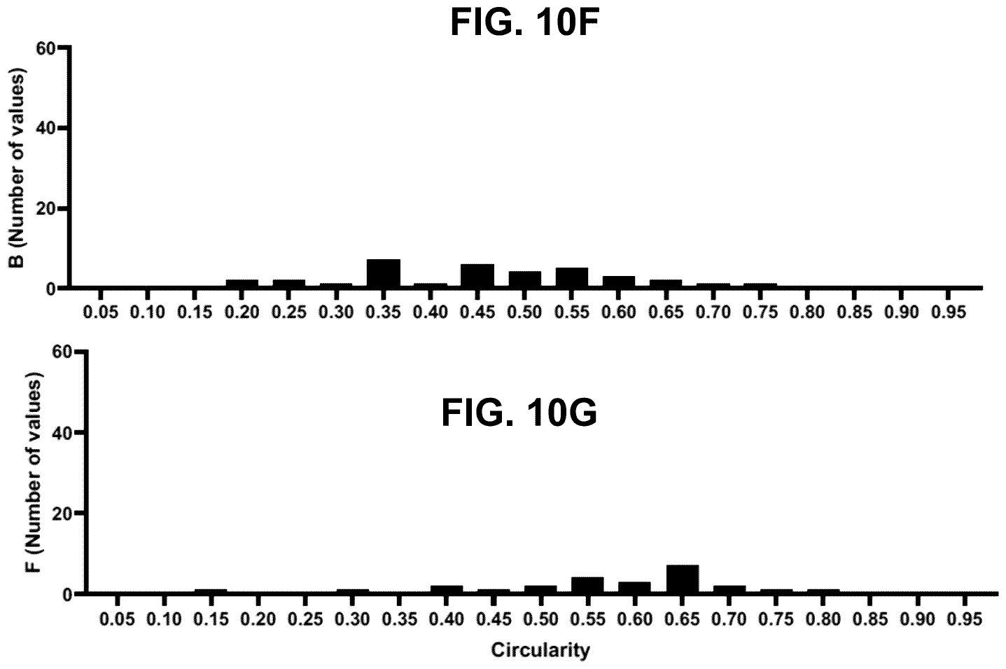

FIGS. 10A-10G illustrate PAEC morphology after 24 hours on discs surface modified with various peptides at 0.2 μmol/cm² concentration according to Test 3. FIG. 10A are stained disk scans of PAECs cultured on 6 different surface modification peptides. FIGS. 10B-10G are graphs of FIJI analysis of circularity frequency distribution. FIJI analysis is a method of quantifying stained disk scans.

FIJI analysis includes splitting channels to get greyscale images, performing thresholding, and analyzing particle shape distribution. Model surface area was 10.1 cm².

The results in FIGS. 8A-10G indicated similar attachment and morphology for surfaces modified with P/G/A/B/F′, P, and G. Surface modification with A was associated with some cells that were elongated but that did not have full stretching as seen in other test conditions, and an increase in rounder cells. Surface modification with B and F was associated with little cell attachment and those that were attached were round with little to no elongation.

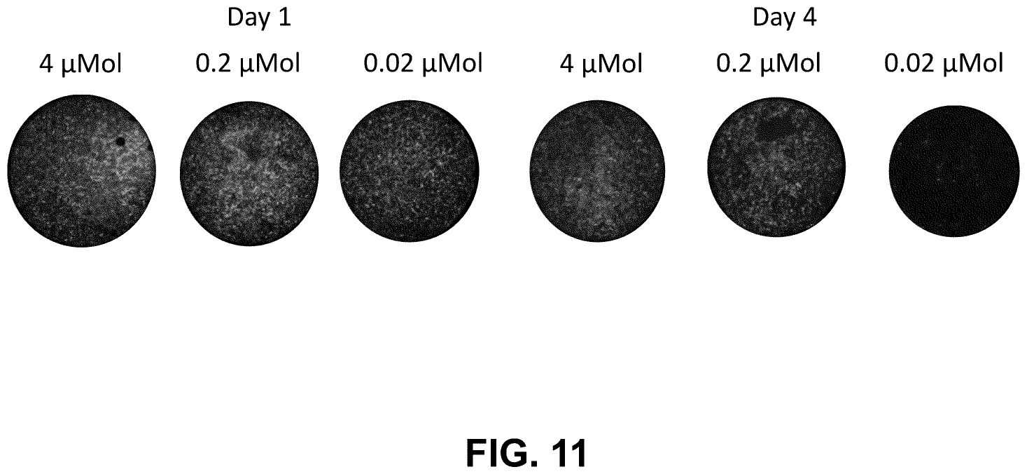

FIG. 11 are images of induced pluripotent stem cell-derived endothelial cells (IPS-ECs) seeded at 50,000/cm² with 4 μMol, 0.2 μMol, and 0.02 μMol concentrations of PABF′ surface modification at day 1 and day 4 after cell seeding. These results indicated greater cell attachment with 0.2 μmol/cm² and 4 μmol/cm² peptide modification concentrations than 0.02 μmol/cm².

The specific embodiments and applications thereof described above are for illustrative purposes only and do not preclude modifications and variations encompassed by the scope of the following claims.

1. A method of modifying a surface of a three-dimensional (3D) article, the surface of the 3D article having a plurality of reactive groups, the method comprising:
(a) immersing at least one part of the 3D article in a buffered solution of functionalized peptides that bind with the reactive groups; and
(b) allowing reaction between the functionalized peptides and the reactive groups on the surface of the 3D article.

2. The method of claim 1, further comprising washing the at least one part of the 3D article to remove unreacted functionalized peptides.

3. The method of claim 1, wherein the reactive groups are thiol-reactive groups.

4. The method of claim 3, wherein the functionalized peptides comprise thiol-functionalized peptides.

5. The method of claim 1, wherein the buffered solution has a pH of about 7.5 to about 10.

6. The method of claim 1, wherein the concentration of functionalized peptides is from about 0.02 μmol per cm² on the surface of the 3D article to about 8.00 μmol per cm² on the surface of the 3D article.

7. The method of claim 1, wherein at least one of the functionalized peptides binds to or has an affinity for at least one of syndecan, integrin, fibronectin, collagen IV, and laminin.

8. The method of claim 1, wherein at least one of the functionalized peptides comprises binding domains for integrin and syndecan.

9. The method of claim 1, wherein the functionalized peptides comprise at least one amino acid sequence selected from the group consisting of CGRDRGDSPY (SEQ ID NO: 1), PHSRNGGGK(GGGERCG)GGRGDSPY (SEQ ID NO: 2) (GGGERCG disclosed as SEQ ID NO: 3), GCREKKRKRLQVQLSIRT (SEQ ID NO: 4), GCREIKVAV (SEQ ID NO: 5), GCREKKTLQPVYEYMVGV (SEQ ID NO: 6), GCREISAFLGIPFAEPPMGPRRFLPPEPKKP (SEQ ID NO: 7), and GGYGGGPG(GPP)5GFOGER(GPP)5GPC (SEQ ID NO: 8).

10. The method of claim 1, further comprising forming the 3D article using 3D printing before immersing.

11. The method of claim 1, further comprising contacting the 3D article with at least one type of cell that has affinity for the functionalized peptides.

12. The method of claim 1, wherein the reactive groups comprise at least one of acrylate, thiol, maleimide, vinyl sulfone, norbornene, acrylamide, acrylonitrile, or methacrylate.

13. The method of claim 12, further comprising immersing the 3D article in cell media prior to contacting with the at least one cell type.

14. The method of claim 13, wherein contacting the 3D article with the at least one cell type comprises immersing the 3D article in a suspension of the cells for at least 1.5 hours.

15. The method of claim 13, wherein contacting the 3D article with the at least one cell type comprises intermittently perfusing the 3D article with a flowing suspension of cells at a flow rate of about 2 mL per minute to about 8 mL per minute.

16. The method of claim 15, wherein contacting the 3D article with the at least one cell type further comprises constantly perfusing the 3D article with a flowing suspension of cells.

17. The method of claim 1, wherein the 3D article comprises a lung scaffold.

18. A three-dimensional (3D) article comprising:
a polymer scaffold having a surface; and
a plurality of peptides covalently bonded to the surface of the polymer scaffold via a cysteine bridge;
wherein at least some of the plurality of peptides bind to or have an affinity for at least two of syndecan, integrin, fibronectin, collagen IV, or laminin.

19. The 3D article of claim 18, wherein at least some of the plurality of peptides bind to or have an affinity for at least three of syndecan, integrin, fibronectin, collagen IV, or laminin.

20. The 3D article of claim 18, wherein the plurality of peptides comprises at least one amino acid sequence CGRDRGDSPY (SEQ ID NO: 1), PHSRNGGGK(GGGERCG)GGRGDSPY (SEQ ID NO: 2) (GGGERCG disclosed as SEQ ID NO: 3), GCREKKRKRLQVOLSIRT (SEQ ID NO: 4), GCREIKVAV (SEQ ID NO: 5), GCREKKTLQPVYEYMVGV (SEQ ID NO: 6), GCREISAFLGIPFAEPPMGPRRFLPPEPKKP (SEQ ID NO: 7), or GGYGGGPG(GPP)5GFOGER(GPP)5GPC (SEQ ID NO: 8).

21. The 3D article of claim 18, further comprising a plurality of biological cells disposed on the surface of the polymer scaffold.

22. The 3D article of claim 21, wherein the biological cells comprise primary adult lung cells.

23. The 3D article of claim 21, wherein the biological cells comprise induced pluripotent stem cells.

24. The 3D article of claim 18, wherein the plurality of peptides is bonded to the surface of the polymer scaffold via a thiol-acrylate Michael addition reaction.

25. The 3D article of claim 24, wherein the polymer scaffold was formed with 3D printing and acrylate groups that participate in the Michael addition reaction were formed at the surface of the polymer scaffold as part of the 3D printing.

26. The 3D article of claim 18, wherein the polymer scaffold comprises a lung scaffold.

27. A method of forming a three-dimensional (3D) article comprising:
forming a lung scaffold using 3D printing, the 3D printing forming a plurality of acrylate groups on an outer surface of the lung scaffold;
immersing at least part of the lung scaffold in a buffered solution suspension of thiol-functionalized peptides that bind to or have an affinity for at least two of syndecan, integrin, fibronectin, collagen IV, or laminin, the buffered solution suspension having a pH of about 7.5 to about 8.5;
washing the lung scaffold to remove unreacted thiol-functionalized peptides;
after washing, conditioning the lung scaffold with cell media; and
after conditioning, seeding biological cells on the outer surface of the lung scaffold using at least one of a static suspension of the biological cells or a flowing suspension of the biological cells.


SEQUENCE LISTING (14 sequences)

SEQ ID NO: 1 - protein, 10 aa, synthetic construct
CGRDRGDSPY

SEQ ID NO: 2 - protein, 17 aa, synthetic construct
  SITE 9: note = Side chain attached to another sequence
PHSRNGGGKGGRGDSPY

SEQ ID NO: 3 - protein, 7 aa, synthetic construct
  SITE 1: note = Attached to another sequence
GGGERCG

SEQ ID NO: 4 - protein, 18 aa, synthetic construct
GCREKKRKRLQVQLSIRT

SEQ ID NO: 5 - protein, 9 aa, synthetic construct
GCREIKVAV

SEQ ID NO: 6 - protein, 18 aa, synthetic construct
GCREKKTLQPVYEYMVGV

SEQ ID NO: 7 - protein, 31 aa, synthetic construct
GCREISAFLGIPFAEPPMGPRRFLPPEPKKP

SEQ ID NO: 8 - protein, 47 aa, synthetic construct
  SITE 26: note = Hydroxyproline
GGYGGGPGGPPGPPGPPGPPGPPGFXGERGPPGPPGPPGPPGPPGPC

SEQ ID NO: 9 - protein, 9 aa, synthetic construct
PHSRNKRGD

SEQ ID NO: 10 - protein, 6 aa, synthetic construct
  SITE 3: note = Hydroxyproline
GFXGER

SEQ ID NO: 11 - protein, 16 aa, synthetic construct
GCRDVPMSMRGGDRCG

SEQ ID NO: 12 - protein, 4 aa, synthetic construct
RGDS

SEQ ID NO: 13 - protein, 5 aa, synthetic construct
IKVAV

SEQ ID NO: 14 - protein, 10 aa, synthetic construct
PSHRNKRGDS